SEQUENCE LISTING (21 sequences)

SEQ ID NO: 1 - DNA, 1377 bp, Mycobacterium tuberculosis
  CDS (4)..(1368)
catatgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagttcatcaccatcactgaagcttg

SEQ ID NO: 2 - protein, 455 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGVHHHH

SEQ ID NO: 3 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagtttaa

SEQ ID NO: 4 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 5 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggattatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagtttaa

SEQ ID NO: 6 - protein, 451 aa, Mycobacterium tuberculosis
MSAVALPRVSGGHDEHGHLEEFRTDPIGIMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 7 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgatccacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagttggcccagcccgcttgcgtgcgctaccgccggcgaacgggagtttaa

SEQ ID NO: 8 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMIHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 9 - DNA, 1356 bp, Mycobacterium tuberculosis
  CDS (1)..(1353)
atgagcgctgttgcactaccccgggtttcgggtggccacgacgaacacggccacctcgaggagttccgcaccgatccgatcgggctgatgcaacgggtccgcgacgaatgcggagacgtcggtaccttccagctggccgggaagcaggtcgtgctgctgtccggctcgcacgccaacgaattcttcttccgggcgggcgacgacgacctggaccaggccaaggcatacccgttcatgacgccgatcttcggcgagggcgtggtgttcgacgccagcccggaacggcgtaaagagatgctgcacaatgccgcgctacgcggcgagcagatgaagggccacgctgccaccatcgaagatcaagtccgacggatgatcgccgactggggtgaggccggcgagatcgatctgctggacttcttcgccgagctgaccatctacacctcctcggcctgcctgatcggcaagaagttccgcgaccagctcgacgggcgattcgccaagctctatcacgagttggagcgcggcaccgacccactagcctacgtcgacccgtatctgccgatcgagagcttccgtcgccgcgacgaagcccgcaatggtctggtggcactggttgcggacatcatgaacggccggatcgccaacccacccaccgacaagagcgaccgtgacatgctcgacgtgctcatcgccgtcaaggctgagaccggcactccccggttctcggccgacgagatcaccggcatgttcatctcgatgatgttcgccggccatcacaccagctcgggtacggcttcgtggacgctgatcgagttgatgcgccatcgcgacgcctacgcggccgtgatcgacgaactcgacgagctgtacggcgacggccgatcggtgagtttccatgcgctgcgccagattccgcagctggaaaacgtgctgaaagagacgctgcgcctgcaccctccgctgatcatcctcatgcgagtggccaagggcgagttcgaggtgcaaggccaccggattcatgagggcgatctggtggcggcctccccggcgatctccaaccggatccccgaagacttccccgatccccacgacttcgtgccagcacgatacgagcagccgcgccaggaagatctgctcaaccgctggacgtggattccgttcggcgccggccggcatcgttgcgtgggggcggcgttcgccatcatgcagatcaaagcgatcttctcggtgttgttgcgcgagtatgagtttgagatggcgcaaccgccagaaagctatcgtaacgaccattcgaagatggtggtgcagatagcccagcccgcttgcgtgcgctaccgccggcgaacgggagtttaa

SEQ ID NO: 10 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQIAQPACVRYRRRTGV

SEQ ID NO: 11 - DNA, 27 bp, Mycobacterium tuberculosis
cgccatatgagcgctgttgcactaccc

SEQ ID NO: 12 - DNA, 46 bp, Mycobacterium tuberculosis
cgcaagcttcagtgatggtgatgaactcccgttcgccggcggtagc

SEQ ID NO: 13 - DNA, 27 bp, Mycobacterium tuberculosis
cgccatatgggctatcgagtcgaagcc

SEQ ID NO: 14 - DNA, 55 bp, Mycobacterium tuberculosis
cgcaagcttcagtgatggtgatgctctcccgtttctcggatggacagtgcctggg

SEQ ID NO: 15 - DNA, 10 bp, Mycobacterium tuberculosis
gaagagggga

SEQ ID NO: 16 - DNA, 74 bp, Mycobacterium tuberculosis
  CDS (24)..(74)
ggcgaagaggggatgccgggctaatgagcctgttgcactaccccgggtttcggggtggccacgacgaacacggc

SEQ ID NO: 17 - protein, 17 aa, Mycobacterium tuberculosis
MSLLHYPGFRGGHDEHG

SEQ ID NO: 18 - protein, 503 aa, Homo sapiens
MALLLAVFAGGSVLGQAMEKVTGGNLLSMLLIACAFTLSLVYLIRLAAGHLVQLPAGVKSPPYIFSPIPFLGHAIAFGKSPIEFLENAYEKYGPVFSFTMVGKTFTYLLGSDAAALLFNSKNEDLNAEDVYSRLTTPVFGKGVAYDVPNPVFLEQKKMLKSGLNIAHFKQHVSIIEKETKEYFESWGESGEKNVFEALSELIILTASHCLHGKEIRSQLNEKVAQLYADLDGGFSHAAWLLPGWLPLPSFRRRDRAHREIKDIFYKAIQKRRQSQEKIDDILQTLLDATYKDGRPLTDDEVAGMLIGLLLAGQHTSSTTSAWMGFFLARDKTLQKKCYLEQKTVCGENLPPLTYDQLKDLNLLDRCIKETLRLRPPIMIMMRMARTPQTVAGYTIPPGHQVCVSPTVNQRLKDSWVERLDFNPDRYLQDNPASGEKFAYVPFGAGRHRCIGENFAYVQIKTIWSTMLRLYEFDLIDGYFPTVNYTTMIHTPENPVIRYKRRSK

SEQ ID NO: 19 - protein, 515 aa, Penicillium italicum
MDLVPLVTGQILGIAYYTTGLFLVSIVLNVIKQLIFYNRKEPPVVFHWIPFIGSTIAYGMDPYQFFFASRAKYGDIFTFILLGKKTTVYLGVEGNEFILNGKLKDVNAEEVYGKLTTPVFGSDVVYDCPNSKLMEQKKFIKYGLSQEALESYVPLIADETNAYIKSSPNFKGQSGTIDLAAAMAEITIFTAARTLQGEEVRSKLTSEFADLFHDLDLGFSPINFMLPWAPLPHNASAIKHTTYARDLSGNYPSATGSWRRRQRRRQDKSKGTDMISNLMRCVYRDGTPIPDKEIAHMMITLLMAGQHSSSAISCWILLRLASQPEMAEKLHAEQIKNLGADLPPLQYKDMDKLPLLRNVIKETLRLHSSIHTLMRKVKNPMPVPGTDFVVPPSHTLLSSPGVTARDERHFRDPLRWDPHRWESRVEVEDSSDTVDYGYGAVSKGTRSPYLPFGAGRHRCIGEKFAYLNLEVIVATLVREFRFFNPEGMEGVPDTDYSSLFSRPVQPATVRWEVRS

SEQ ID NO: 20 - protein, 280 aa, Triticum sp.
MQPISVIFPYLPIPAHRRRDQARTRLAEIFATIIKSRKASGQSEEDMLQCFIDSKYKNGRQTTESEVTGLLIAALFAGQHTSSITSTWTGAYLLKFQQYFAEAVEEQKEVMKRHGDKIDHDILAEMDVLYRCIKEALRLHPPLIMLLRQSHSDFSVTTREGKEFDIPKGHIVATSPAFANRLPHIFKNPDSYDPDRFAAGREEDKVAGAFSYISFGGGRHGCLGEPFAYLQIKAIWTHLLRNFEFELVSPFPENDWNAMVVGIKGEVMVSYKRRKLVVDN

SEQ ID NO: 21 - protein, 517 aa, Candida albicans
MAIVETVIDGINYFLSGVPFVYNLVWQYLYSLRKDRAPLVFYWIPWFGSAASYGQQPYEFFESCRQKYGDVFSFMLLGKIMTVYLGPKGHEFVFNAKLSDVSAEEAYKHLTTPVFGTGVIYDCPNSRLMEQKKFAKFALTTDSFKRYVPKIREEILNYFVTDESFKLKEKTHGVANVMKTQPEITIFTASRSLFGDEMRRIFDRSFAQLYSDLDKGFTPINFVFPNLPLPHYWRRDAAQKKISATYMKEIKSRRERGDIDPNRDLIDSLLIHSTYKDGVKMTDQEIANLLIGILMGGQHTSASTSAWFLLHLGEKPHLQDVIYQEVVELLKEKGGDLNDLTYEDLQKLPSVNNTIKETLRMHMPLHSIFRKVTNPLRIPETNYIVPKGHYVLVSPGYAHTSERYFDNPEDFDPTRWDTAAAKANSVSFNSSDEVDYGFGKASKGVSSPYLPFSGGRHRCIGEQFAYVQLGTILTTFVYNLRWTIDGYKVPDPDYSSMVVLPTEPAEIIWEKRETCMF

(19) United States
(12) Patent Application Publication
(10) Pub. No.: US 2002/0052031 A1
(43) Pub. Date: May 2, 2002

(54) Mycobacterium tuberculosis CYP51 polypeptides and nucleic acids and therapeutic and screening methods relating to same

(76) Inventors: Michael R. Waterman, Nashville, TN (US); Aouatef Bellamine, Nashville, TN (US)

Correspondence Address: JENKINS & WILSON, PA, 3100 TOWER BLVD, SUITE 1400, DURHAM, NC, 27707, US

(21) Appl. No.: 09/909,903

(22) Filed: Jul. 20, 2001

Related U.S. Application Data
(62) Division of application No. 09/345,218, filed on Jun. 30, 1999, now abandoned.

(51) Int. Cl. C12N 9/02; C07H 21/04; C12N 5/06; C07K 16/40

(52) U.S. Cl. 435/189; 536/23.2; 530/388.26; 435/338

(57) ABSTRACT

A cytochrome p450 14α-demethylase enzyme isolated from Mycobacterium tuberculosis designated as MT CYP51. A crystalline form of MT CYP51 is also disclosed. Nucleic acid molecules encoding MT CYP51 are also disclosed. Recombinant host cells, recombinant nucleic acids and recombinant proteins are also disclosed, along with methods of producing each. Isolated and purified antibodies to MT CYP51, and methods of producing the same, are also disclosed. MT CYP51 is characterized as having 14α-demethylase biological activity. Thus, therapeutic and drug screening methods pertaining to this activity are also disclosed.

### GRANT STATEMENT

This work was supported by NIH grants R37 GM37942 and ES 00267-32. Thus, the U.S. Government has certain rights in the invention.

### TECHNICAL FIELD

The present invention relates generally to isolated and purified polypeptides and to isolated and purified nucleic acids encoding such polypeptides. More particularly, the present invention relates to isolated and purified Mycobacterium tuberculosis CYP51 polypeptides and isolated and purified nucleic acid molecules encoding the same.

Table of Abbreviations

| | |
|---|---|
| BMP | Bone morphogenetic protein |
| BSA | Bovine serum albumin |
| CDR(s) | Complementarity determining region(s) |
| CYP450 | Cytochrome P450 |
| CYP45014DM | Cytochrome P450 14α-demethylase |
| CYP51 | Cytochrome P450 14α-demethylase |
| DHL | 24,25-dihydrolanosterol |
| Fdr | Escherichia coil flavodoxin reductase |
| Fdx | Mycobacterium tuberculosis ferredoxin |
| Fld | Escherichia coil flavodoxin |
| Fnr | Spinach ferredoxin reductase |
| GC-MS | Gas chromatography-Mass spectroscopy |
| HAT | Cell culture media comprising hypoxanthine, |
| | aminopterin, and thymidine |
| HPLC | High pressure liquid chromatography |
| KLH | Keyhole limpet hemocyanin |
| MT | Mycobacterium tuberculosis |
| NMR | Nuclear magnetic resonance |
| PCR | Polymerase chain reaction |
| SRS | Substrate recognition sequence |

### BACKGROUND ART

The cytochrome P450 enzyme cytochrome P450 14α-demethylase (CYP45014DM) catalyzes 14α-demethylation of different sterols via three successive oxidations at the C-32 methyl group. CYP45014DM is thus involved in cholesterol, ergosterol and phytosterol biosynthesis in animals, fungi and plants, respectively. In animals, for example, the demethylation reaction catalyzed by CYP45014DM results in the formation of formic acid and 4,4-dimethyl-5α cholesta-8,14-,24-trien-3β-ol from lanosterol.

Although the function of this enzyme has been conserved between different species, CYP45014DM substrate specificity is narrow. It has been reported that yeast and animals utilize lanosterol and dihydrolanosterol while filimentus fungi utilize eburicol (24-methylene lanosterol). Plant CYP45014DM's, however, use only obtusifoliol as a substrate. Currently, this enzyme is the only CYP450 enzyme found in three different phyla: animals, fungi and plants.

Thus, CYP45014DM enzymes are present in a wide variety of organisms. But, to date no bacterial CYP45014DM has been fully characterized. Moreover, given the key sterol metabolic pathway in which this enzyme is involved, there is a continuing need in the art for further characterization of CYP45014DM enzymes in general. In particular, there remains a continuing need for the characterization of substrate specificity and the elucidation of crystalline structures.

### SUMMARY OF THE INVENTION

The present invention contemplates an isolated and purified Mycobacterium tuberculosis (MT) cytochrome P450 14α-demethylase (MT P45014DM or MT CYP51) polypeptide. Preferably, the polypeptide is biologically active. More preferably, a polypeptide of the invention is a recombinant polypeptide. Even more preferably, a polypeptide of the present invention comprises the amino acid residue sequence of any of SEQ ID NO's:2, 4, 6, 8 and 10.

The present invention also provides an isolated and purified polynucleotide that encodes a biologically active MT CY51 polypeptide. More preferably, a polynucleotide of the present invention comprises a polynucleotide designated MT CYP51. Even more preferred, a polynucleotide of the present invention encodes a polypeptide comprising the amino acid residue sequence of any of SEQ ID NO's:2, 4, 6, 8 and 10. Most preferably, an isolated and purified polynucleotide of the invention comprises the nucleotide base sequence of any of SEQ ID NO's:1, 3, 5, 7 and 9.

In another embodiment, the present invention provides an antibody immunoreactive with an MT CYP51 polypeptide as described above. Also contemplated by the present invention are antibodies immunoreactive with homologues or biologically equivalent MT CYP51. Preferably, an antibody of the invention is a monoclonal antibody. Even more preferably, the MT CYP51 polypeptide against which the monoclonal antibody is detected comprises the amino acid residue sequence of any of SEQ ID NO's:2, 4, 6, 8 and 10.

In another aspect, the present invention contemplates a process of producing an antibody immunoreactive with an MT CYP51 polypeptide as described above, the process comprising the steps of (a) transfecting a recombinant host cell with a polynucleotide that encodes a biologically active MT CYP51 polypeptide; (b) culturing the host cell under conditions sufficient for expression of the polypeptide; (c) purifying the polypeptide; and (d) raising the antibody to the polypeptide. SEQ ID NO's:1-10 set forth preferred nucleotide and amino acid sequences. Preferably, the host cell is transfected with a polynucleotide of any of SEQ ID NO's:1, 3, 5, 7 and 9. Even more preferably, the present invention provides an antibody prepared according to the process described above. Also contemplated by the present invention is the use of homologues or biologically equivalent polynucleotides and polypeptides to produce antibodies.

Alternatively, the present invention provides a process of detecting a MT CYP51 polypeptide as described above, wherein the process comprises immunoreacting the polypeptide with an antibody prepared according to the process described above to form an antibody-polypeptide conjugate, and detecting the conjugate.

In yet another embodiment, the present invention contemplates a process of detecting a messenger RNA transcript that encodes a MT CYP51 polypeptide as described above, wherein the process comprises hybridizing the messenger RNA transcript with a polynucleotide sequence that encodes that polypeptide to form a duplex; and detecting the duplex. Alternatively, the present invention provides a process of detecting a DNA molecule that encodes a MT CYP51 polypeptide as described above, wherein the process comprises hybridizing DNA molecules with a polynucleotide that encodes a biologically active MT CYP51 polypeptide to form a duplex; and detecting the duplex.

In another aspect, the present invention contemplates an assay kit for detecting the presence of a MT CYP51 polypeptide in a biological sample, where the kit comprises a first container containing a first antibody capable of immunoreacting with a biologically active MT CYP51 polypeptide, with the first antibody present in an amount sufficient to perform at least one assay. Preferably, an assay kit of the invention further comprises a second container containing a second antibody that immunoreacts with the first antibody. More preferably, the antibodies used in an assay kit of the present invention are monoclonal antibodies. Even more preferably, the first antibody is affixed to a solid support. More preferably still, the first and second antibodies comprise an indicator, and, preferably, the indicator is a radioactive label or an enzyme.

In an alternative aspect, the present invention provides an assay kit for detecting the presence, in biological samples, of a MT CYP51 polypeptide, the kits comprising a first container that contains a second polynucleotide identical or complementary to a segment of at least 10 contiguous nucleotide bases of a polynucleotide that encodes a biologically active MT CYP51 polypeptide.

In another embodiment, the present invention contemplates an assay kit for detecting the presence, in a biological sample, of an antibody immunoreactive with a MT CYP51 polypeptide, the kit comprising a first container containing a biologically active MT CYP51 polypeptide that immunoreacts with the antibody, with the polypeptide present in an amount sufficient to perform at least one assay.

In still a further embodiment, this invention pertains to therapeutic methods based upon the modulation of the biological activity of MT CYP51 as described herein.

Thus, a key aspect of this invention pertains to the discovery that the MT CYP51 nucleic acid encodes the functional MT CYP51 polypeptide. Preferred nucleic acid and amino acid sequences for MT CYP51 are described in SEQ ID NO's:1-10.

The foregoing aspects and embodiments have broad utility given the biological significance of the CYP450 enzymes, as is known in the art. By way of example, the foregoing aspects and embodiments are useful in the preparation of screening assays and assay kits that are used to identify compounds that affect or modulate MT CYP51 biological activity, or that are used to detect the presence of the polypeptides and nucleic acids of this invention in biological samples. Additionally, it is well known that isolated and purified polypeptides have utility as feed additives for livestock and further polynucleotides encoding the polypeptides are thus useful in producing the polypeptides.

It is thus an object of the present invention to provide a purified and isolated biologically active MT CYP51 polypeptide.

It is another object of the present invention is to provide crystals of sufficient quality to obtain a determination of the three-dimensional structure of a biologically active MT CYP51 polypeptide to high resolution, preferably to the resolution of less than about 2.0 angstroms.

It is still another object of the present invention to provide a starting material for use in the determination of the structure of members of the CYP450 superfamily of enzymes.

It is yet another object of the present invention to provide a starting material for use in the rational design of drugs which mimic or inhibit the action of the CYP450 superfamily of enzymes.

Some of the aspects and objects of the invention having been stated hereinabove, other aspects and objects will become evident as the description proceeds, when taken in connection with the accompanying Drawings and Laboratory Examples as best described hereinbelow.

### BRIEF DESCRIPTION OF THE DRAWINGS

FIG. 1 is a schematic depicting the activity of CYP45014DM in sterol biosynthesis. Note that the 14α-methyl group which is removed by this enzyme is indicated in each substrate.

FIG. 2 is a schematic depicting alignment of the amino acid sequence of Mycobacterium tuberculosis (MT) CYP51 gene product with Homo sapiens (H.s); Penicilium italicum (P.i.); Triticum aevestivum (T.a.); and Candida albicans (C.a.) sequences. The boxed residues correspond to the substrate recognition sequence (SRS) elements, expected to be found in all cytochrome P450 enzymes. The arrow corresponds to the cysteine heme-ligand. No homology is observed in the N-terminal sequence between the other CYP51 isoforms and the MT CYP51 gene product. This is because the MT CYP51 gene product is a soluble protein and the others are anchored in cellular membranes.

FIG. 3 pertains to sterol molecule structures and DHL 14α-demethylation.

FIG. 3A depicts sterol molecule structures: (1) lanosterol, (2) cycloartenol, (3) parkeol, (4) DHL, (5) zymosterol and (6) obtusifoliol.

FIG. 3B is a reaction schematic for DHL 14α-demethylation, that is, conversion of DHL to 4,4-dimethyl-5α-cholesta-8,14-diene-3β-ol in the presence of MT P45014DM, NADPH and molecular oxygen.

FIG. 3C is a line graph depicting GLC profile of overnight conversion of 2 mg DHL. The E. coli Fld/Fdr system was used as P450 electron donor. The peaks at 16.38 min and 17 min correspond to the DHL and metabolite retention times, respectively.

FIG. 4 depicts sequence and absorbence characteristics of the MT CYP51 gene and gene product.

FIG. 4A depicts a potential Shine-Dalgarno sequence (shadowed box) of the MT CYP51 gene, the ATG is represented in bold character.

FIG. 4B depicts absorbance of purified MT P45014DM (400 pmol), absolute oxidized form (regular trace), sodium hydrosulfite reduced form (dashed trace). The inset shows the α and β bands for the oxidized and the reduced forms.

FIG. 4C depicts differential CO-reduced P450 spectrum of purified MT P45014DM (400 pmol).

FIG. 4D depicts (1) silver staining and (2) immunoblot analysis using 1 pmol and 0.4 pmol of purified MT P45014DM, respectively. MT P45014DM antibody prepared with TiterMax@gold™ as adjuvant was used at 1:5000 dilution. Protein G-horseradish peroxidase conjugate (BIO-RAD) was used as a second antibody and ECL kit for detection.

FIG. 5 depicts comparison of MT P45014DM activities supported by either Fld/Fdr or Fdx/Fnr. [24-³H]DHL was converted overnight at 30° C. by 1 nmole of MT P45014DM with either 20 nmoles Fld and 2 nmoles Fdr (panel A) or 20 nmoles Fdx and 2 nmoles Fnr (panel B) (30). Peaks S and P correspond to DHL and its 14α-demethylated product, respectively. Peaks U are unidentified products. MT P45014DM used in this experiment was further purified by HLPC (BIOCAD®/Sprint, PerSeptive Biosystems, Inc., Framingham, Mass.) using Poros HS and HQ columns (PerSeptive Biosystems, Inc., Framingham, Mass.). The HS flow-through is loaded on an HQ column and eluted using a NaCl gradient (150 to 500 mM).

FIG. 6 depicts MT P45014DM binding spectra.

FIG. 6A depicts MT P45014DM type I binding spectrum for obtusifoliol (100 nM-5 μM).

FIG. 6B depicts double reciprocal plot for obtusifoliol (●), DHL (▴) and lanosterol (▪) binding with 10 μM of MT P45014DM.

FIG. 6C. MT P45014DM type II binding spectrum in presence of clotrimazole (500 nM-100 μM).

FIG. 6D. Double reciprocal plot for clotrimazole (∘), ketoconazole (●) and fluconazole (▴) binding with 5 μM of MT P45014DM.

FIG. 7 is a western blot analysis of 0.4 pmol of purified recombinant MT P45014DM (lanes 1 and 3) and 100 μmg of MT cytosolic fraction (lanes 2 and 4) using complete (lanes 1 and 2) and the depleted (lanes 3 and 4) antisera. The antiserum raised using Freund's adjuvant was purified using a MT P45014DM sepharose affinity column followed by batch chromatography with the same resin (Gough, N. M. & Adams, J. M. (1978) Biochemistry 17: 5560-6). The antiserum was depleted by overnignt incubation with 6 nmole of purified MT P45014DM at 4° C.

### DETAILED DESCRIPTION OF THE INVENTION

Disclosed herein is the cloning and isolation of a MT CYP51 gene and a polypeptide encoded by this gene. During the isolation and cloning of the gene, four histidine codons were added at the 3′ end. The polypeptide was expressed in E. coli at a level of about 500 nmol of soluble CYP51 per liter of culture. The polypeptide was subsequently purified using a Ni⁺² affinity column, and the purified enzyme showed oxidized, reduced and reduced-CO spectra typical for a biologically active CYP450.

The purified polypeptide was biologically active in that it was able to convert dihydrolanosterol to its 14α-demethylated product. This reaction was inhibited by ketoconazole. The purified biologically active polypeptide demonstrated substrate specificity for lanosterol, dihydrolanosterol and obtusifoliol. Particularly, dihydrolanosterol and obtusifoliol were metabolized by the purified biologically active polypeptide. The disclosure of the present invention demonstrates the existence of a CYP450 14α-demethylase in a fourth phylum, bacteria. Unlike eukaryotic forms, the bacterial CYP450 14α-demethylase is a soluble CYP450.

### Definitions and Techniques Affecting Gene Products and Genes

The present invention concerns DNA segments, isolatable from bacterial cells, which are free from genomic DNA and which are capable of conferring CYP450 14α-demethylase biological activity in a recombinant host cell when incorporated into the recombinant host cell. DNA segments capable of conferring CYP450 14α-demethylase biological activity may encode complete MT CYP51 polypeptides, cleavage products and biologically active functional domains thereof.

The terms “MT CYP51 protein”, “MT CYP51 polypeptide”, “MT CYP51 gene product”, “MT CYP51”, “MT CYP45014DM protein”, “MT CYP45014DM polypeptide”, and “MT CYP45014DM”, as used in the specification and in the claims, are meant to be synonymous and to refer to proteins having amino acid sequences which are substantially identical to the respective native MT CYP45014DM amino acid sequences and which have CYP450 14α-demethylase biological activity or are capable of cross-reacting with an anti-MT CYP51 antibody raised against MT CYP51. Such sequences are disclosed herein. The terms “MT CYP51 protein”, “MT CYP51 polypeptide”, “MT CYP51 gene product”, “MT CYP51”, “MT CYP45014DM protein”, “MT CYP45014DM polypeptide”, and “MT CYP45014DM” also include analogs of MT P45014DM molecules which exhibit at least some biological activity in common with native MT CYP45014DM.

Furthermore, those skilled in the art of mutagenesis will appreciate that other analogs, as yet undisclosed or undiscovered, may be used to construct MT CYP51 analogs. There is no need for a “MT CYP51 protein”, “MT CYP51 polypeptide”, “MT CYP51 gene product”, “MT CYP51”, “MT CYP45014DM protein”, “MT CYP45014DM polypeptide”, and “MT CYP45014DM” to comprise all, or substantially all, of the amino acid sequence encoded by the native MT CYP51 gene. Shorter or longer sequences are anticipated to be of use in the invention. The term “fragment” refers to any subject polypeptide having an amino acid residue sequence shorter than that of a polypeptide whose amino acid residue sequence is shown herein.

The terms “MT CYP51 gene”, “MT CYP51 gene sequence” and “MT CYP51 gene segment” refer to any DNA sequence that is substantially identical to a DNA sequence encoding a MT CYP51 polypeptide or MT CYP51 as defined above. The terms also refer to RNA, or antisense sequences, compatible with such DNA sequences. A “MT CYP51 gene”, “MT CYP51 gene sequence” and “MT CYP51 gene segment” may also comprise any combination of associated control sequences.

The term “substantially identical”, when used to define either a MT CYP51 or MT CYP51 amino acid sequence, or a MT CYP51 gene or MT CYP51 nucleic acid sequence, means that a particular sequence, for example, a mutant sequence, varies from the sequence of a natural MT CYP51 by one or more deletions, substitutions, or additions, the net effect of which is to retain at least some of biological activity of MT CYP51. Alternatively, DNA analog sequences are “substantially identical” to specific DNA sequences disclosed herein if: (a) the DNA analog sequence is derived from coding regions for the natural MT CYP51 or from the natural MT CYP51 gene; or (b) the DNA analog sequence is capable of hybridization of DNA sequences of (a) under moderately stringent conditions and which encode biologically active MT CYP51; or (c) the DNA sequences are degenerative as a result of the genetic code to the DNA analog sequences defined in (a) and/or (b).

Substantially identical analog proteins will be greater than about 60% identical to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequences.

### Percent Similarity

Percent similarity may be determined, for example, by comparing sequence information using the GAP computer program, available from the University of Wisconsin Geneticist Computer Group. The GAP program utilizes the alignment method of Needleman et al. 1970, as revised by Smith et al. 1981. Briefly, the GAP program defines similarity as the number of aligned symbols (i.e. nucleotides or amino acids) which are similar, divided by the total number of symbols in the shorter of the two sequences. The preferred default parameters for the GAP program include: (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps.

The term “homology” describes a mathematically based comparison of sequence similarities which is used to identify genes or proteins with similar functions or motifs. Accordingly, the term “homology” is synonymous with the term “similarity” and “percent similarity” as defined above. Thus, the phrases “substantial homology” or “substantial similarity” have similar meanings.

### Nucleic Acid Sequences

In certain embodiments, the invention concerns the use of MT CYP51 genes and gene products that include within their respective sequences a sequence which is essentially that of the MT CYP51 gene, or the corresponding protein. The term “a sequence essentially as that of MT CYP51 or MT CYP51 gene”, means that the sequence substantially corresponds to a portion of a MT CYP51 or MT CYP51 gene and has relatively few bases or amino acids (whether DNA or protein) which are not identical to those of a MT CYP51 or MT CYP51 gene, (or a biologically functional equivalent of, when referring to proteins). The term “biologically functional equivalent” is well understood, in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of a MT CYP51 or MT CYP51 gene, will be sequences which are “essentially the same”.

MT CYP51 encoding nucleic acid sequences which have functionally equivalent codons are also covered by the invention. The term “functionally equivalent codon” is used herein to refer to codons that encode the same amino acid, such as the six codons for arginine or serine. Thus, when referring to the sequence examples presented in SEQ ID NO's:1-10 applicants contemplate substitution of functionally equivalent codons of Table 1 into the sequence examples of SEQ ID NO's:1-10. Thus, applicants are in possession of amino acid and nucleic acids sequences which include such substitutions but which are not set forth herein in their entirety for convenience.

The term “functionally equivalent codon” is also used herein to refer to codons that encode biologically equivalent amino acids (see Table 1). Thus, when referring to the sequence examples presented in SEQ ID NO's:1-10 applicants contemplate substitution from Table 1 of codons that encode biologically equivalent amino acids as described herein into the sequence examples of SEQ ID NO's:1-10. Thus, applicants are in possession of amino acid and nucleic acids sequences which include such substitutions but which are not set forth herein in their entirety for convenience.

TABLE I
Functionally Equivalent Codons.

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUG CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UGU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood that amino acid and nucleic acid sequences may include additional residues, such as additional—or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences which may, for example, include various non-coding sequences flanking either of the 5′ or 3′ portions of the coding region or may include various internal sequences which are known to occur within genes.

The present invention also encompasses the use of DNA segments which are complementary, or essentially complementary, to the sequences set forth in the specification. Nucleic acid sequences which are “complementary” are those which are base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment in question under relatively stringent conditions such as those described herein. A particular example of a contemplated complementary nucleic acid segment is an antisense oligonucleotide.

Nucleic acid hybridization will be affected by such conditions as salt concentration, temperature, or organic solvents, in addition to the base composition, length of the complementary strands, and the number of nucleoticie base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. (See, e.g., Wetmur & Davidson, 1968).

Probe sequences may also hybridize specifically to duplex DNA under certain conditions to form triplex or other higher order DNA complexes. The preparation of such probes and suitable hybridization conditions are well known in the art.

As used herein, the term “DNA segment” refers to a DNA molecule which has been isolated free of total genomic DNA of a particular species. Furthermore, a DNA segment encoding a MT CYP51 refers to a DNA segment which contains MT CYP51 coding sequences, yet is isolated away from, or purified free from, total genomic DNA of Mycobacterium tuberculosis. Included within the term “DNA segment” are DNA segments and smaller fragments of such segments, and also recombinant vectors, including, for example, plasmids, cosmids, phages, viruses, and the like.

Similarly, a DNA segment comprising an isolated or purified MT CYP51 gene refers to a DNA segment including MT CYP51 coding sequences isolated substantially away from other naturally occurring genes or protein encoding sequences. In this respect, the term “gene” is used for simplicity to refer to a functional protein, polypeptide or peptide encoding unit. “Isolated substantially away from other coding sequences” means that the gene of interest, in this case, the MT CYP51 gene, forms the significant part of the coding region of the DNA segment, and that the DNA segment does not contain large portions of naturally-occurring coding DNA, such as large chromosomal fragments or other functional genes or coding regions. Of course, this refers to the DNA segment as originally isolated, and does not exclude genes or coding regions later added to the segment by the hand of man.

In particular embodiments, the invention concerns isolated DNA segments and recombinant vectors incorporating DNA sequences which encode a MT CYP51 that includes within its amino acid sequence the amino acid sequence of any of SEQ ID NO's:2, 4, 6, 8 and 10.

It will also be understood that this invention is not limited to the particular nucleic acid and amino acid sequences of SEQ ID NOS:1 and 2. Recombinant vectors and isolated DNA segments may therefore variously include the MT CYP51-encoding region itself, include coding regions bearing selected alterations or modifications in the basic coding region, or include encoded larger polypeptides which nevertheless include MT CYP51-encoding regions or may encode biologically functional equivalent proteins or peptides which have variant amino acid sequences.

In certain embodiments, the invention concerns isolated DNA segments and recombinant vectors which encode a protein or peptide that includes within its amino acid sequence an amino acid sequence essentially as set forth in of any of SEQ ID NO's:2, 4, 6, 8 and 10. Naturally, where the DNA segment or vector encodes a full length MT CYP51 gene product, the most preferred sequence is that which is essentially as set forth in any of SEQ ID NO's:1, 3, 5, 7 and 9 and which encode a protein that exhibits CYP450 14α-demethylase metabolic activity in, for example, bacterial cells, as may be determined by, for example, sterol metabolism assays as disclosed herein.

The term “a sequence essentially as set forth in of any of SEQ ID NO's:2, 4, 6, 8 and 10” means that the sequence substantially corresponds to a portion of any of SEQ ID NO's:2, 4, 6, 8 and 10 and has relatively few amino acids which are not identical to, or a biologically functional equivalent of, the amino acids of any of SEQ ID NO's:2, 4, 6, 8 and 10. The term “biologically functional equivalent” is well understood in the art and is further defined in detail herein. Accordingly, sequences, which have between about 70% and about 80%; or more preferably, between about 81% and about 90%; or even more preferably, between about 91% and about 99%; of amino acids which are identical or functionally equivalent to the amino acids of any of SEQ ID NO's:2, 4, 6, 8 and 10, will be sequences which are “essentially as set forth in any of SEQ ID NO's:2, 4, 6, 8 and 10”.

In certain other embodiments, the invention concerns isolated DNA segments and recombinant vectors that include within their sequence a nucleic acid sequence essentially as set forth in any of SEQ ID NO's:1, 3, 5, 7 and 9. The term “essentially as set forth in any of SEQ ID NO's:1, 3, 5, 7 and 9” is used in the same sense as described above and means that the nucleic acid sequence substantially corresponds to a portion of any of SEQ ID NO's:1, 3, 5, 7 and 9, respectively, and has relatively few codons which are not identical, or functionally equivalent, to the codons of any of SEQ ID NO's:1, 3, 5, 7 and 9, respectively. Again, DNA segments which encode gene products exhibiting CYP450 14α-demethylase activity of the MT CYP51 gene product will be most preferred. The term “functionally equivalent codon” is used herein to refer to codons that encode the same amino acid, such as the six codons for arginine or serine, and also to refer to codons that encode biologically equivalent amino acids (see Table 1).

The nucleic acid segments of the present invention, regardless of the length of the coding sequence itself, may be combined with other DNA sequences, such as promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, nucleic acid fragments may be prepared which include a short stretch complementary to any of SEQ ID NO's:1, 3, 5, 7 and 9, such as about 10 nucleotides, and which are up to 10,000 or 5,000 base pairs in length, with segments of 3,000 being preferred in certain cases. DNA segments with total lengths of about 1,000, 500, 200, 100 and about 50 base pairs in length are also contemplated to be useful.

The DNA segments of the present invention encompass biologically functional equivalent MT CYP51 proteins and peptides. Such sequences may rise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein or to test MT CYP51 mutants in order to examine CYP450 14α-demethylase activity at the molecular level.

If desired, one may also prepare fusion proteins and peptides, e.g., where the MT CYP51 coding region is aligned within the same expression unit with other proteins or peptides having desired functions, such as for purification or immunodetection purposes (e.g., proteins which may be purified by affinity chromatography and enzyme label coding regions, respectively).

Recombinant vectors form important further aspects of the present invention. Particularly useful vectors are contemplated to be those vectors in which the coding portion of the DNA segment is positioned under the control of a promoter. The promoter may be in the form of the promoter which is naturally associated with the MT CYP51 gene, e.g., in MT cells, as may be obtained by isolating the 5′ non-coding sequences located upstream of the coding segment, for example, using recombinant cloning and/or PCR technology, in connection with the compositions disclosed herein.

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a MT CYP51 gene in its natural environment. Such promoters may include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology, for example, see Sambrook et al., 1989, specifically incorporated herein by reference. The promoters employed may be constitutive, or inducible, and can be used under the appropriate conditions to direct high level expression of the introduced DNA segment, such as is advantageous in the large-scale production of recombinant proteins or peptides. Appropriate promoter systems contemplated for use in high-level expression include, but are not limited to, the vaccina virus promoter and the baculovirus promoter, which are more fully described below.

In an alternative embodiment, the present invention provides an expression vector comprising a polynucleotide that encodes a MT CYP51 polypeptide having CYP450 14α-demethylase metabolic activity. Also preferably, an expression vector of the present invention comprises a polynucleotide that encodes human MT CYP51. More preferably, an expression vector of the present invention comprises a polynucleotide that encodes a polypeptide comprising the amino acid residue sequence of any of SEQ ID NO's:2, 4, 6, 8 and 10. More preferably, an expression vector of the present invention comprises a polynucleotide comprising the nucleotide base sequence of any of SEQ ID NO's:1, 3, 5, 7 and 9. Even more preferably, an expression vector of the invention comprises a polynucleotide operatively linked to an enhancer-promoter. More preferably still, an expression vector of the invention comprises a polynucleotide operatively linked to a prokaryotic promoter. Alternatively, an expression vector of the present invention comprises a polynucleotide operatively linked to an enhancer-promoter that is a eukaryotic promoter, and the expression vector further comprises a polyadenylation signal that is positioned 3′ of the carboxy-terminal amino acid and within a transcriptional unit of the encoded polypeptide.

In yet another embodiment, the present invention provides a recombinant host cell transfected with a polynucleotide that encodes a MT CYP51 polypeptide having CYP450 14α-demethylase metabolic activity. SEQ ID NO s:1-10 set forth exemplary nucleotide and amino acid sequences from MT. Also contemplated by the present invention are homologous or biologically equivalent MT CYP51 polynucleotides and polypeptides. Preferably, a recombinant host cell of the present invention is transfected with the polynucleotide sequence of any of SEQ ID NO's:1, 3, 5, 7 and 9.

In another aspect, a recombinant host cell of the present invention is a prokaryotic host cell. Preferably, a recombinant host cell of the invention is a bacterial cell, preferably a strain of Escherichia coli. More preferably, a recombinant host cell comprises a polynucleotide under the transcriptional control of regulatory signals functional in the recombinant host cell, wherein the regulatory signals appropriately control expression of the MT CYP51 polypeptide in a manner to enable all necessary transcriptional and post-transcriptional modification.

In yet another embodiment, the present invention contemplates a process of preparing an MT CYP51 polypeptide comprising transfecting a cell with polynucleotide that encodes an MT CYP51 polypeptide having CYP450 14α-demethylase activity to produce a transformed host cell; and maintaining the transformed host cell under biological conditions sufficient for expression of the polypeptide. More preferably, the host cell is a prokaryotic cell. More preferably, the prokaryotic cell is a bacterial cell of the HMS174 strain of Escherichia coli. Even more preferably, a polynucleotide transfected into the transformed cell comprises the nucleotide base sequence of any of SEQ ID NO's:1, 3, 5, 7 and 9. SEQ ID NO's:1-10 set forth nucleotide and amino acid sequences for MT. Also contemplated by the present invention are homologues or biologically equivalent CYP51 polynucleotides and polypeptides found in other bacterial species.

As mentioned above, in connection with expression embodiments to prepare recombinant MT CYP51 proteins and peptides, it is contemplated that longer DNA segments will most often be used, with DNA segments encoding the entire MT CYP51 protein, functional domains or cleavage products thereof, being most preferred. However, it will be appreciated that the use of shorter DNA segments to direct the expression of MT CYP51 peptides or epitopic core regions, such as may be used to generate anti-MT CYP51 antibodies, also falls within the scope of the invention.

DNA segments which encode peptide antigens from about 15 to about 50 amino acids in length, or more preferably, from about 15 to about 30 amino acids in length are contemplated to be particularly useful. DNA segments encoding peptides will generally have a minimum coding length in the order of about 45 to about 150, or to about 90 nucleotides. DNA segments encoding full length proteins preferably have a coding length on the order of about 1353 nucleotides for a protein in accordance with any of SEQ ID NO's:2, 4, 6, 8 and 10.

Naturally, the present invention also encompasses DNA segments which are complementary, or essentially complementary, to the sequence set forth in any of SEQ ID NO's:1, 3, 5, 7 and 9. The terms “complementary” and “essentially complementary” are defined above. Excepting flanking regions, and allowing for the degeneracy of the genetic code, sequences which have between about 70% and about 80%; or more preferably, between about 81% and about 90%; or even more preferably, between about 91% and about 99%; of nucleotides which are identical or functionally equivalent (i.e. encoding the same amino acid) of nucleotides of any of SEQ ID NO's:1, 3, 5, 7 and 9, will be sequences which are “essentially as set forth in any of SEQ ID NO's:1, 3, 5, 7 and 9”. Sequences which are essentially the same as those set forth in any of SEQ ID NO's:1, 3, 5, 7 and 9 may also be functionally defined as sequences which are capable of hybridizing to a nucleic acid segment containing the complement of any of SEQ ID NO's:1, 3, 5, 7 and 9 under relatively stringent conditions. Suitable relatively stringent hybridization conditions are described herein and will be well known to those of skill in the art.

### Biologically Functional Equivalents

As mentioned above, modification and changes may be made in the structure of the MT CYP51 proteins and peptides described herein and still obtain a molecule having like or otherwise desirable characteristics. For example, certain amino acids may be substituted for other amino acids in a protein structure without appreciable loss of interactive capacity with lanosterol, dihydrolanosterol and other substrates. Since it is the interactive capacity and nature of a protein that defines that protein's biological activity, certain amino acid sequence substitutions can be made in a protein sequence (or, of course, its underlying DNA coding sequence) and nevertheless obtain a protein with like or even countervailing properties (e.g., antagonistic v. agonistic). It is thus contemplated by the inventors that various changes may be made in the sequence of the MT CYP51 proteins and peptides (or underlying DNA) without appreciable loss of their biological utility or activity.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged. This is the case in the present invention, where if any changes, for example, SRS elements or cysteine-hem ligands, could result in a loss of an aspect of the utility of the resulting peptide for the present invention.

Amino acid substitutions, such as those which might be employed in modifying the MT CYP51 proteins and peptides described herein, are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (-0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (-2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA, taking into consideration also that the genetic code is degenerate and that two or more codons may code for the same amino acid.

### Sequence Modification Techniques

Modifications to the MT CYP51 proteins and peptides described herein may be carried out using techniques such as site directed mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 30 nucleotides in length is preferred, with about 5 to 10 residues on both sides of the junction of the sequence being altered.

In general, the technique of site-specific mutagenesis is well known in the art as exemplified by publications (e.g., Adelman et al., 1983). As will be appreciated, the technique typically employs a phage vector which exists in both a single stranded and double stranded form. Typical vectors useful in site-directed mutagenesis include vectors such as the M13 phage (Messing et al., 1981). These phage are readily commercially available and their use is generally well known to those skilled in the art. Double stranded plasmids are also routinely employed in site directed mutagenesis which eliminates the step of transferring the gene of interest from a plasmid to a phage.

In general, site-directed mutagenesis in accordance herewith is performed by first obtaining a single-stranded vector or melting apart the two strands of a double stranded vector which includes within its sequence a DNA sequence which encodes, for example, the MT CYP51 gene. An oligonucleotide primer bearing the desired mutated sequence is prepared, generally synthetically, for example by the method of Crea et al. (1978). This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected gene using site-directed mutagenesis is provided as a means of producing potentially useful MT CYP51 or other biologically active species and is not meant to be limiting as there are other ways in which sequence variants of these peptides may be obtained. For example, recombinant vectors encoding the desired genes may be treated with mutagenic agents to obtain sequence variants (see, e.g., a method described by Eichenlaub, 1979) for the mutagenesis of plasmid DNA using hydroxylamine.

### Other Structural Equivalents

Applicants also contemplate that sterically similar compounds may be formulated to mimic the key portions of the peptide structure. Such compounds may be used in the same manner as the peptides of the invention and hence are also functional equivalents. The generation of a structural and functional equivalent may be achieved by the techniques of modeling and chemical design known to those of skill in the art. It will be understood that all such sterically similar constructs fall within the scope of the present invention.

### Introduction of Gene Products

Where the gene itself is employed to introduce the gene products, a convenient method of introduction will be through the use of a recombinant vector which incorporates the desired gene, together with its associated control sequences. The preparation of recombinant vectors is well known to those of skill in the art and described in many references, such as, for example, Sambrook et al. (1989), specifically incorporated herein by reference.

In vectors, it is understood that the DNA coding sequences to be expressed, in this case those encoding the MT CYP51 gene products, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One may also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of the specific gene (i.e., the MT CYP51 promoter for MT CYP51 ) will be preferred, there is no reason why other control sequences could not be employed, so long as they are compatible with the genotype of the cell into which gene products are being introduced. Thus, one may mention other useful promoters by way of example, including, e.g., an SV40 early promoter, a long terminal repeat promoter from retrovirus, an actin promoter, a heat shock promoter, a metallothionein promoter, and the like.

As is known in the art, a promoter is a region of a DNA molecule typically within about 100 nucleotide pairs in front of (upstream on the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the Hind III site toward the Bgl I site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

Where the MT CYP51 gene itself is employed it will be most convenient to simply use the wild type MT CYP51 gene directly. However, it is contemplated that certain regions of the MT CYP51 gene may be employed exclusively without employing the entire isolated wild type MT CYP51 gene. It is proposed that it will ultimately be preferable to employ the smallest region needed to impart CYP450 14α-demethylase metabolic activity so that one is not introducing unnecessary DNA into cells which receive an MT CYP51 gene construct. Techniques well known to those of skill in the art, such as the use of restriction enzymes, will allow for the generation of small regions of the MT CYP51 gene. The ability of these regions to impart CYP450 14α-demethylase metabolic activity can easily be determined by the assays reported in the Examples. In general, techniques for assessing CYP450 144α-demethylase metabolic activity are well known in the art.

### Generation of Antibodies

In still another embodiment, the present invention provides an antibody immunoreactive with a polypeptide of the present invention. Preferably, an antibody of the invention is a monoclonal antibody. Means for preparing and characterizing antibodies are well known in the art (See, e.g., Antibodies: A Laboratory Manual, E. Howell and D. Lane, Cold Spring Harbor Laboratory, 1988).

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polygonal antibodies.

As is well known in the art, a given polypeptide or polynucleotide may vary in its immunogenicity. It is often necessary therefore to couple the immunogen (e.g., a polypeptide or polynucleotide) of the present invention) with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier protein are well known in the art and include glutaraldehyde, m-maleimidobenzoyl-N-hydroxysuccinimide ester, carbodiimide and bis-biazotized benzidine.

As is also well known in the art, immunogencity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used for the production of polyclonal antibodies varies, inter alia, upon the nature of the immunogen as well as the animal used for immunization. A variety of routes can be used to administer the immunogen (subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

In another aspect, the present invention contemplates a process of producing an antibody immunoreactive with MT CYP51 polypeptide, the process comprising the steps of (a) transfecting recombinant host cells with a polynucleotide that encodes that polypeptide; (b) culturing the host cells under conditions sufficient for expression of the polypeptide; (c) recovering the polypeptide; and (d) preparing antibodies to the polypeptide. Preferably, the MT CYP:1 polypeptide possesses CYP450 14α-demethylase biological activity. Even more preferably, the present invention provides antibodies prepared according to the process described above.

A monoclonal antibody of the present invention can be readily prepared through use of well-known techniques such as those exemplified in U.S. Pat. No. 4,196,265, herein incorporated by reference. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptides. The selected clones can then be propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis ). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immuncreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides and polynucleotide of the invention can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

### Detecting a Polynucleotide or a Polypeptide of the Present Invention

Alternatively, the present invention provides a process of detecting a polypeptide of the present invention, wherein the process comprises immunoreacting the polypeptides with antibodies prepared according to the process described above to form antibody-polypeptide conjugates, and detecting the conjugates.

In yet another embodiment, the present invention contemplates a process of detecting messenger RNA transcripts that encode a polypeptide of the present invention, wherein the process comprises hybridizing the messenger RNA transcripts with polynucleotide sequences that encode the polypeptide to form duplexes; and detecting the duplex. Alternatively, the present invention provides a process of detecting DNA molecules that encode a polypeptide of the present invention, wherein the process comprises hybridizing DNA molecules with a polynucleotide that encodes that polypeptide to form duplexes; and detecting the duplexes.

### Screening Assays for a Polypeptide of the Present Invention

The present invention provides a process of screening a biological sample for the presence of a MT CYP51 polypeptide. Preferably, the MT CYP51 polypeptide possesses CYP450 14α-demethylase biological activity. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide.

In accordance with a screening assay process, a biological sample is exposed to an antibody immunoreactive with the polypeptide whose presence is being assayed. Typically, exposure is accomplished by forming an admixture in a liquid medium that contains both the antibody and the candidate polypeptide. Either the antibody or the sample with the polypeptide can be affixed to a solid support (e.g., a column or a microtiter plate).

The biological sample is exposed to the antibody under biological reaction conditions and for a period of time sufficient for antibody-polypeptide conjugate formation. Biological reaction conditions include ionic composition and concentration, temperature, pH and the like.

Ionic composition and concentration can range from that of distilled water to a 2 molal solution of NaCl. Preferably, osmolality is from about 100 mosmols/l to about 400 mosmols/l and, more preferably from about 200 mosmols/l to about 300 mosmols/l. Temperature preferably is from about 4° C. to about 100° C., more preferably from about 15° C. to about 50° C. and, even more preferably from about 25° C. to about 40° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for antibody-polypeptide conjugate formation and that the conditions do not adversely affect either the antibody or the polypeptide.

Exposure time will vary inter alia with the biological conditions used, the concentration of antibody and polypeptide and the nature of the sample (e.g., fluid or tissue sample). Means for determining exposure time are well known to one of ordinary skill in the art. Typically, where the sample is fluid and the concentration of polypeptide in that sample is about 10⁻¹⁰ M, exposure time is from about 10 minutes to about 200 minutes.

The presence of polypeptide in the sample is detected by detecting the formation and presence of antibody-polypeptide conjugates. Means for detecting such antibody-antigen (e.g., ligand-polypeptide) conjugates or complexes are well known in the art and include such procedures as centrifugation, affinity chromatography and the like, binding of a secondary antibody to the antibody-candidate receptor complex.

In one embodiment, detection is accomplished by detecting an indicator affixed to the antibody. Exemplary and well known such indicators include radioactive labels (e.g., ³²P, ¹²⁵I, ¹⁴C), a second antibody or an enzyme such as horse radish peroxidase. Means for affixing indicators to antibodies are well known in the art. Commercial kits are available.

### Screening Assay for Anti-Polypeptide Antibody

In another aspect, the present invention provides a process of screening a biological sample for the presence of antibodies immunoreactive with a MT CYP51 polypeptide. Preferably the MT CYP51 polypeptide possesses CYP450 14α-demethylase biological activity. In accordance with such a process, a biological sample is exposed to an MT CYP51 polypeptide under biological conditions and for a period of time sufficient for antibody-polypeptide conjugate formation and the formed conjugates are detected.

### Screening Assay for Polynucleotide That Encodes a MT CYP51 Polypeptide of the Present Invention

A DNA molecule and, particularly a probe molecule, can be used for hybridizing as an oligonucleotide probe to a DNA source suspected of encoding an MT CYP51 polypeptide of the present invention. Preferably the MT CYP51 polypeptide possesses CYP450 14α-demethylase biological activity. The probing is usually accomplished by hybridizing the oligonucleotide to a DNA source suspected of possessing an MT CYP51 gene. In some cases, the probes constitute only a single probe, and in others, the probes constitute a collection of probes based on a certain amino acid sequence or sequences of the polypeptide and account in their diversity for the redundancy inherent in the genetic code.

A suitable source of DNA for probing in this manner is capable of expressing a polypeptide of the present invention and can be a genomic library of a cell line of interest. Alternatively, a source of DNA can include total DNA from the cell line of interest. Once the hybridization process of the invention has identified a candidate DNA segment, one confirms that a positive clone has been obtained by further hybridization, restriction enzyme mapping, sequencing and/or expression and testing.

Alternatively, such DNA molecules can be used in a number of techniques including their use as: (1) tools to detect normal and abnormal DNA sequences in DNA derived from cells; (2) means for detecting and isolating other members of the polypeptide family and related polypeptides from a DNA library potentially containing such sequences; (3) primers for hybridizing to related sequences for the purpose of amplifying those sequences; (4) primers for altering native MT CYP51 DNA sequences; as well as other techniques which rely on the similarity of the DNA sequences to those of the DNA segments herein disclosed.

As set forth above, in certain aspects, DNA sequence information provided by the invention allows for the preparation of relatively short DNA (or RNA) sequences (e.g., probes) that specifically hybridize to encoding sequences of a selected MT CYP51 gene. In these aspects, nucleic acid probes of an appropriate length are prepared based on a consideration of the encoding sequence for a polypeptide of this invention. The ability of such nucleic acid probes to specifically hybridize to other encoding sequences lend them particular utility in a variety of embodiments. Most importantly, the probes can be used in a variety of assays for detecting the presence of complementary sequences in a given sample. However, other uses are envisioned, including the use of the sequence information for the preparation of mutant species primers, or primers for use in preparing other genetic constructions.

To provide certain of the advantages in accordance with the invention, a preferred nucleic acid sequence employed for hybridization studies or assays includes probe sequences that are complementary to at least a 14 to 40 or so long nucleotide stretch of a nucleic acid sequence of the present invention, such as; that shown in any of SEQ ID NO's:1, 3, 5, 7 and 9. A size of at least 14 nucleotides in length helps to ensure that the fragment is of sufficient length to form a duplex molecule that is both stable and selective. Molecules having complementary sequences over stretches greater than 14 bases in length are generally preferred, though, to increase stability and selectivity of the hybrid, and thereby improve the quality and degree of specific hybrid molecules obtained. One will generally prefer to design nucleic acid molecules having gene-complementary stretches of 14 to 20 nucleotides, or even longer where desired. Such fragments can be readily prepared by, for example, directly synthesizing the fragment by chemical means, by application of nucleic acid reproduction technology, such as the PCR technology of U.S. Pat. No. 4,683,202, herein incorporated by reference, or by introducing selected sequences into recombinant vectors for recombinant production.

Accordingly, a nucleotide sequence of the present invention can be used for its ability to selectively form duplex molecules with complementary stretches of the gene. Depending on the application envisioned, one employs varying conditions of hybridization to achieve varying degrees of selectivity of the probe toward the target sequence. For applications requiring a high degree of selectivity, one typically employs relatively stringent conditions to form the hybrids. For example, one selects relatively low salt and/or high temperature conditions, such as provided by 0.02M-0.15M salt at temperatures of about 50° C. to about 70° C. including particularly temperatures of about 55° C., about 60° C. and about 65° C. Such conditions are particularly selective, and tolerate little, if any, mismatch between the probe and the template or target strand.

Of course, for some applications, for example, where one desires to prepare mutants employing a mutant primer strand hybridized to an underlying template or where one seeks to isolate polypeptide coding sequences from related species, functional equivalents, or the like, less stringent hybridization conditions are typically needed to allow formation of the heteroduplex. Under such circumstances, one employs conditions such as 0.15M-0.9M salt, at temperatures ranging from about 20° C. to about 55° C., including particularly temperatures of about 25° C., about 37° C., about 45° C., and about 50° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

In certain embodiments, it is advantageous to employ a nucleic acid sequence of the present invention in combination with an appropriate means, such as a label, for determining hybridization. A wide variety of appropriate indicator means are known in the art, including radioactive, enzymatic or other ligands, such as avidin/biotin, which are capable of giving a detectable signal. In preferred embodiments, one likely employs an enzyme tag such a urease, alkaline phosphatase or peroxidase, instead of radioactive or other environmentally undesirable reagents. In the case of enzyme tags, calorimetric indicator substrates are known which can be employed to provide a means visible to the human eye or spectrophotometrically, to identify specific hybridization with complementary nucleic acid-containing samples.

In general, it is envisioned that the hybridization probes described herein are useful both as reagents in solution hybridization as well as in embodiments employing a solid phase. In embodiments involving a solid phase, the sample containing test DNA (or RNA) is adsorbed or otherwise affixed to a selected matrix or surface. This fixed, single-stranded nucleic acid is then subjected to specific hybridization with selected probes under desired conditions. The selected conditions depend inter alia on the particular circumstances based on the particular criteria required (depending, for example, on the G+C contents, type of target nucleic acid, source of nucleic acid, size of hybridization probe, etc.). Following washing of the hybridized surface so as to remove nonspecifically bound probe molecules, specific hybridization is detected, or even quantified, by means of the label.

### Assay Kits

In another aspect, the present invention contemplates assay kits for detecting the presence of a polypeptide of the present invention in biological samples, where the kits comprise a first container containing a first antibody capable of immunoreacting with the polypeptide, with the first antibody present in an amount sufficient to perform at least one assay. Preferably, the assay kits of the invention further comprise a second container containing a second antibody that immunoreacts with the first antibody. More preferably, the antibodies used in the assay kits of the present invention are monoclonal antibodies. Even more preferably, the first antibody is affixed to a solid support. More preferably still, the first and second antibodies comprise an indicator, and, preferably, the indicator is a radioactive label or an enzyme.

The present invention also contemplates a kit for screening agents. Such a kit can contain a polypeptide of the present invention. The kit can contain reagents for detecting an interaction between an agent and a receptor of the present invention. The provided reagent can be radiolabeled. The kit can contain a known radiolabelled agent capable of binding or interacting with a receptor of the present invention.

In an alternative aspect, the present invention provides assay kits for detecting the presence, in biological samples, of a polynucleotide that encodes a polypeptide of the present invention, the kits comprising a first container that contains a second polynucleotide identical or complementary to a segment of at least 10 contiguous nucleotide bases of, as a preferred example, any of SEQ ID NO's:1, 3, 5, 7 and 9.

In another embodiment, the present invention contemplates assay kits for detecting the presence, in a biological sample, of antibodies immunoreactive with a polypeptide of the present invention, the kits comprising a first container containing a MT CYP51 polypeptide, that immunoreacts with the antibodies, with the polypeptide present in an amount sufficient to perform at least one assay. Preferably, the MT CYP51 polypeptide possesses CYP450 14α-demethylase biological activity. The reagents of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media or a microscope slide. When the reagent provided is a dry powder, the powder can be reconstituted by the addition of a suitable solvent. The solvent can be provided.

### Screening Assays

In yet another aspect, the present invention contemplates a process of screening substances for their ability to affect or modulate the biological activity of CYP51 enzymes, and preferably, the biological activity of MT CYP51. More preferably, the present invention contemplates a process of screening substances for their ability to affect or modulate the biological activity of MT CYP51 to thereby affect or modulate MT growth or infection. Utilizing the methods and compositions of the present invention, screening assays for the testing of candidate substances can be derived. A candidate substance is a substance which potentially can promote but preferably inhibits the biological activity of MT CYP51 to thereby affect or modulate the MT growth or infection.

An exemplary method of screening candidate substances for their ability to modulate CYP51 biological activity comprises the steps of: (a) establishing replicate test and control samples that comprise a biologically active MT CYP51 polypeptide; (b) administering a candidate substance to test sample but not the control sample; (c) measuring the biological activity of the MT CYP51 polypeptide in the test and the control samples; and (d) determining that the candidate substance modulates MT CYP51 biological activity if the biological activity of the MT CYP51 polypeptide measured for the test sample is greater or less than the biological activity of the MT CYP51 polypeptide level measured for the control sample.

The replicate test and control samples can further comprise a cell that expresses a biologically active CYP51 polypeptide. The present invention thus also contemplates a recombinant cell line suitable for use in this method.

A screening assay of the present invention generally involves determining the ability of a candidate substance to modulate CYP51 biological activity in a target cell, such as the screening of candidate substances to identify those that modulate, i.e. inhibit or promote, CYP51 biological activity. Preferably, the CYP51 polypeptide comprises a MT CYP51 polypeptide. Target cells can be either naturally occurring cells known to contain a polypeptide of the present invention (e.g. MT cells) or transformed cell produced in accordance with a process of transformation set forth hereinbefore.

As is well known in the art, a screening assay provides a cell under conditions suitable for testing the modulation of CYP51 biological activity. These conditions include but are not limited to pH, temperature, tonicity, the presence of relevant metabolic factors (e.g., metal ions such as for example Ca⁺⁺, growth factor, interleukins, or colony stimulating factors), and relevant modifications to the polypeptide such as glycosylation or prenylation. It is contemplated that a polypeptide of the present invention can be expressed and utilized in a prokaryotic or eukaryotic cell. The host cell can also be fractionated into sub-cellular fractions where CYP45014DM enzymatic substrates can be found. For example, cells expressing the polypeptide can be fractionated into the nuclei, the endoplasmic reticulum, vesicles, or the membrane surfaces of the cell.

pH is preferably from about a value of 6.0 to a value of about 8.0, more preferably from about a value of about 6.8 to a value of about 7.8 and, most preferably about 7.4. In a preferred embodiment, temperature is from about 20° C. to about 50° C., more preferably from about 30° C. to about 40° C. and, even more preferably about 37° C. Osmolality is preferably from about 5 milliosmols per liter (mosm/L) to about 400 mosm/l and, more preferably from about 200 milliosmols per liter to about 400 mosm/l and, even more preferably from about 290 mosm/L to about 310 mosm/L. The presence of factors can be required for the proper testing of CYP51 biological activity modulation in specific cells. Such factors include, for example, the presence and absence (withdrawal) of growth factor, interleukins, colony stimulating factors and/or reductase systems for CYP450 enzymes. U.S. Pat. No. 5,645,999 also describes exemplary screening assays, and the entire contents of U.S. Pat. No. 5,645,999 are herein incorporated by reference.

In one embodiment, a screening assay is designed to be capable of discriminating candidate substances having selective ability to interact with one or more of the polypeptides of the present invention but which polypeptides are without a substantially overlapping activity with another of those polypeptides identified herein. Exemplary assays including genetic screening assays and molecular biology screens such as a yeast two-hybrid screen which will effectively identify CYP51-interacting genes important for CYP450 14α-demethylase metabolism modulation or other CYP51-mediated biological activity. One version of the yeast two-hybrid system has been described (Chien et al., 1991, Proc. Natl. Acad. Sci. USA, 88:9578-9582) and is commercially available from Clontech (Palo Alto, Calif.).

### Rational Drug Design Methods

The knowledge of the structure of the CYP450 family of proteins, and particularly the MT CYP 51 polypeptide of the present invention, provides a means of investigating the mechanism of action of these proteins in a subject. For example, binding of these proteins to various substrate molecules can be predicted by various computer models. Upon discovering that such binding in fact takes place, knowledge of the protein structure then allows chemists to design and attempt to synthesize small molecules which mimic the functional binding of the CYP450-family protein to the substrate. This is the method of “rational” drug design.

Use of the isolated and purified MT CYP51 polypeptide of the present invention, particularly in crystalline form, in rational drug design is thus contemplated in accordance with the present invention. Additional rational drug design techniques are described in U.S. Pat. Nos. 5,834,228 and 5,872,011, the entire contents of which are herein incorporated by reference.

Thus, a method of identifying modulators of CYP450 enzymes by rational drug design is contemplated in accordance with the present invention. The method comprising the steps of designing a potential modulator for a CYP450 enzyme that will form non-covalent bonds with amino acids in the CYP450 enzyme substrate binding site based upon the crystal structure of a MT CYP51 polypeptide; synthesizing the modulator; and determining whether the potential modulator modulates the activity of a CYP450 enzyme. Modulators are synthesized using techniques disclosed herein and as are known in the art. The determination of whether the modulator modulates the biological activity of a CYP450 enzyme is made in accordance with the screening methods disclosed hereinabove.

For example, a contemplated modulator comprises a peptide modulator, also referred to herein as a subject peptide, and can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv Enzymol, 32:221-96,1969; Fields et al., Int. J. Peptide Protein Res., 35:161-214,1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as exemplary, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above may be reacted to form their corresponding cyclic peptides. An exemplary method for cyclizing peptides is described by Zimmer et al., Peptides 1992, pp. 393-394, ESCOM Science Publishers, B. V., 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Purification of the resulting peptides is accomplished using conventional procedures, such as preparative HPLC using gel permeation, partition and/or ion exchange chromatography. The choice of appropriate matrices and buffers are well known in the art and so are not described in detail herein.

### Therapeutic Methods

The candidate drugs and other therapeutic agents screened in accordance with the method of the present invention are contemplated to be useful in the treatment of warm-blooded vertebrates. Therefore, the invention concerns mammals and birds.

Contemplated is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

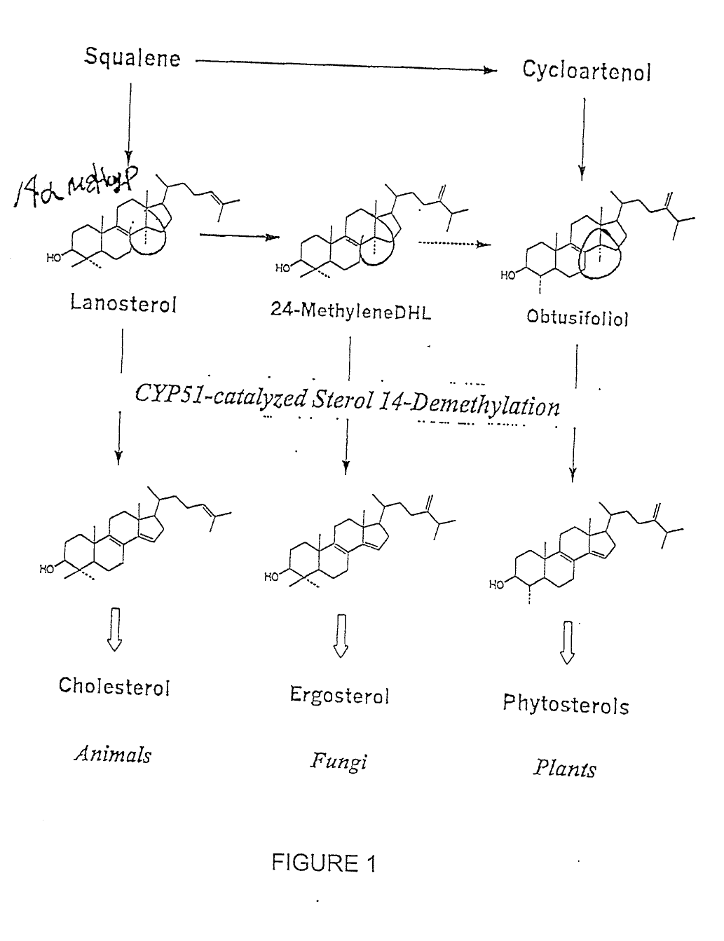

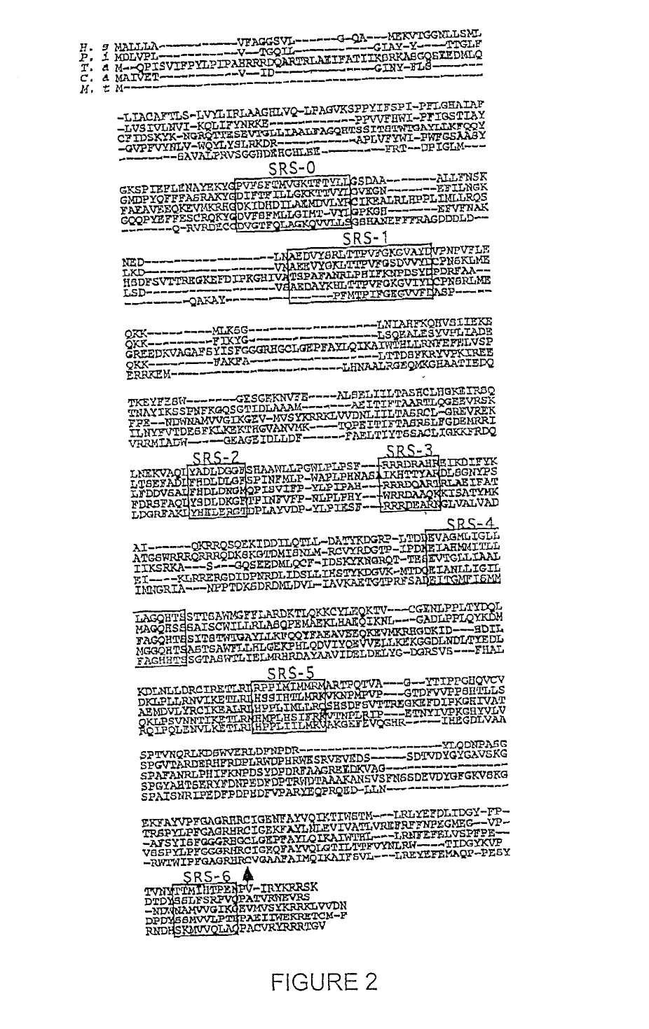

As used herein, the terms “MT CYP51 activity” and “MT CYP51 biological activity” are meant to be synonymous and are meant to refer to any biological activity of MT CYP51. For example, the MT CYP51 gene product is characterized herein as having CYP450 14α-demethylase metabolic activity and this metabolic activity is contemplated by the use of the term “biological activity”. Given that CYP450 14α-demethylase catalyzes an essential step in sterol metabolism (see FIG. 1), modulation of the metabolic activity of the MT CYP51 thus modulates growth and/or infection of MT in a subject.

In view of the foregoing, a therapeutic method is contemplated according to the present invention. The therapeutic method comprises administering to a subject a substance that modulates MT CYP51 biological activity to thereby modulate growth or infection by MT in the subject. Such a substance may be identified according to the screening assay set forth above. A preferred subject is a vertebrate subject. A preferred example of a vertebrate subject is a mammal. A preferred example of a mammal is a human.

Thus, the method may comprise treating a patient suffering from a disorder associated with CYP51 biological activity by administering to the patient an effective CYP51 activity-modulating amount of a substance identified according to the screening assay described above. By the term “modulating”, it is contemplated that the substance can optionally promote or inhibit the activity of CYP51, depending on the disorder to be treated.

Since MT is the major pathogen associated with the disease tuberculosis, a method of treating tuberculosis is contemplated in accordance with the present invention and is described in detail in the Examples below. The contemplated method comprises administering a therapeutically effective amount of a MT CYP51 gene product activity modulator to a subject in need thereof. Preferably, the MT CYP51 activity modulator is in a pharmaceutically acceptable form.

The CYP51 modulators described herein, including MT CYP51 modulators, are thus adapted for administration as pharmaceutical compositions. Formulation and dose preparation techniques have been described in the art, see for example, those described in U.S. Pat. No. 5,326,902 issued to Seipp et al. on Jul. 5, 1994, U.S. Pat. No. 5,234,933 issued to Marnett et al. on Aug. 10, 1993, and PCT Publication WO 93/25521 of Johnson et al. published Dec. 23, 1993, the entire contents of each of which are herein incorporated by reference.

For the purposes described above, the identified substances may normally be administered systemically or partially, usually by oral or parenteral administration. The doses to be administered are determined depending upon age, body weight, symptom, the desired therapeutic effect, the route of administration, and the duration of the treatment etc. In a human adult, the doses per person per administration are generally between 1 mg and 500 mg, by oral administration, up to several times per day, and between 1 mg and 100 mg, by parenteral administration up to several times per day. Since the doses to be used depend upon various conditions, as mentioned above, there may be a case in which doses are lower than or greater than the ranges specified above.

Solid compositions for oral administration include compressed tablets, pills, dispersible powders, capsules, and granules. In such compositions, one or more of the active substance(s) is or are, admixed with at least one inert diluent (lactose, mannitol, glucose, hydroxypropylcellulose, microcrystalline cellulose, starch, polyvinylpyrrolidone, magnesium metasilicate alminate, etc.). The compositions may also comprise, as is normal practice, additional substances other than inert diluents: e.g. lubricating agents (magnesium stearate, etc.), disintegrating agents (cellulose, calcium glycolate etc.), and assisting agent for dissolving (glutamic acid, aspartic acid, etc.) stabilizing agent (lactose etc.). The tablets or pills may, if desired, be coated with gastric or enteric material (sugar, gelatin, hydroxypropylcellulose or hydroxypropylmethyl cellulose phthalate, etc.). Capsules include soft ones and hard ones.

Liquid compositions for oral administration include pharmaceutically-acceptable emulsions, solutions, suspensions, syrups and elixirs. In such compositions, one or more of the active substance(s) is or are admixed with inert diluent(s) commonly used in the art (purified water, ethanol etc.). Besides inert diluents, such compositions may also comprise adjuvants (wetting agents, suspending agents, etc.), sweetening agents, flavoring agents, perfuming agents and preserving agents.

Other compositions for oral administration include spray compositions which may be prepared by known methods and which comprise one or more of the active substance(s). Spray compositions may comprise additional substances other than inert diluents: e.g. preserving agents (sodium sulfite, etc.), isotonic buffer (sodium chloride, sodium citrate, citric acid, etc.). For preparation of such spray compositions, for example, the method described in U.S. Pat. Nos. 2,868,691 or 3,095,355 may be used.

Injections for parenteral administration include sterile aqueous or non-aqueous solution, suspensions and emulsions. In such compositions, one or more of active substance(s) is or are admixed with at least one inert aqueous diluent(s) (distilled water for injection, physiological salt solution etc.) or inert non-aqueous diluent(s) (propylene glycol, polyethylene glycol, olive oil, ethanol, POLYSOLBATE80® etc.). Injections may comprise additional other than inert diluents: e.g. preserving agents, wetting agents, emulsifying agents, dispersing agents, stabilizing agents (lactose, etc.), assisting agents such as for dissolving (glutamic acid, aspartic acid, etc.). They may be sterilized, for example, by filtration through a bacteria-retaining filter, by incorporation of sterilizing agents in the compositions or by irradiation. They also be manufactured in the form of sterile solid compositions, for example, by freeze-drying, and which can be dissolved in sterile water or some other sterile diluents for injection immediately before use.

Other compositions for administration include liquids for external use, and endermic linaments (ointment, etc.), suppositories and pessaries which comprise one or more of the active substance(s) and may be prepared by known methods.

A preferred CYP51 modulator has the ability to substantially interact with a CYP51 in solution at modulator concentrations of less than one (1) micro molar (μM), preferably less than 0.1 μM, and more preferably less than 0.01 μM. By “substantially” is meant that at least a 50 percent reduction in CYP51 biological activity is observed by modulation in the presence of the CYP51 modulator, and at 50% reduction is referred to herein as an IC50 value.

A therapeutically effective amount of a CYP51 modulator of this invention in the form of a monoclonal antibody, or fragment thereof, is typically an amount such that when administered in a physiologically tolerable composition is sufficient to achieve a plasma concentration of from about 0.01 microgram (ug) per milliliter (ml) to about 100 ug/ml, preferably from about 1 ug/ml to about 5 ug/ml, and usually about 5 ug/ml.

The therapeutic compositions containing a CYP51 activity modulator of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier or vehicle.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

Monoclonal Antibodies

The present invention describes, in one embodiment, MT CYP51 modulators in the form of monoclonal antibodies which immunoreact with MT CYP51 and bind the MT CYP51 to modulate metabolic activity as described herein. The invention also describes above cell lines which produce the antibodies, methods for producing the cell lines, and methods for producing the monoclonal antibodies.

A monoclonal antibody of this invention comprises antibody molecules that 1) immunoreact with isolated MT CYP51, and 2) bind to the MT CYP51 to modulate its biological function.

The term “antibody or antibody molecule” in the various grammatical forms is used herein as a collective noun that refers to a population of immunoglobulin molecules and/or immunologically active portions of immunoglobulin molecules, i.e., molecules that contain an antibody combining site or paratope. An “antibody combining site” is that structural portion of an antibody molecule comprised of heavy and light chain variable and hypervariable regions that specifically binds antigen.

Exemplary antibodies for use in the present invention are intact immunoglobulin molecules, substantially intact immunoglobulin molecules, single chain immunoglobulins or antibodies, those portions of an immunoglobulin molecule that contain the paratope, including those portions known in the art as Fab, Fab′, F(ab′)2 and F(v), and also referred to as antibody fragments. Indeed, it is contemplated to be within the scope of the present invention that a monovalent modulator may optionally be is used in the present method. Thus, the terms “modulate”, “modulating”, and “modulator” are meant to be construed to encompass such promotion.

The phrase “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g., a bispecific monoclonal antibody. Methods of producing a monoclonal antibody, a hybridoma cell, or a hybridoma cell culture are described above.

It is also possible to determine, without undue experimentation, if a monoclonal antibody has the same (i.e., equivalent) specificity (immunoreaction characteristics) as a monoclonal antibody of this invention by ascertaining whether the former prevents the latter from binding to a preselected target molecule. If the monoclonal antibody being tested competes with the monoclonal antibody of the invention, as shown by a decrease in binding by the monoclonal antibody of the invention in standard competition assays for binding to the target molecule when present in the solid phase, then it is likely that the two monoclonal antibodies bind to the same, or a closely related, epitope.

Still another way to determine whether a monoclonal antibody has the specificity of a monoclonal antibody of the invention is to pre-incubate the monoclonal antibody of the invention with the target molecule with which it is normally reactive, and then add the monoclonal antibody being tested to determine if the monoclonal antibody being tested is inhibited in its ability to bind the target molecule. If the monoclonal antibody being tested is inhibited then, in all likelihood, it has the same, or functionally equivalent, epitopic specificity as the monoclonal antibody of the invention.

An additional way to determine whether a monoclonal antibody has the specificity of a monoclonal antibody of the invention is to determine the amino acid residue sequence of the CDR regions of the antibodies in question. Antibody molecules having identical, or functionally equivalent, amino acid residue sequences in their CDR regions have the same binding specificity. “CDRs” (complementarity determining regions) mean the three subregions of the light or heavy chain variable regions which have hypervariable sequences and form loop structures that are primarily responsible for making direct contact with antigen. Antibody molecules having identical, or functionally equivalent, amino acid residue sequences in their CDR regions have the same binding specificity. Methods for sequencing polypeptides are well known in the art.

The immunospecificity of an antibody, its target molecule binding capacity, and the attendant affinity the antibody exhibits for the epitope, are defined by the epitope with which the antibody immunoreacts. The epitope specificity is defined at least in part by the amino acid residue sequence of the variable region of the heavy chain of the immunoglobulin that comprises the antibody, and in part by the light chain variable region amino acid residue sequence. Use of the terms “having the binding specificity of” or “having the binding preference of” indicates that equivalent monoclonal antibodies exhibit the same or similar immunoreaction (binding) characteristics and compete for binding to a preselected target molecule.

Humanized monoclonal antibodies offer particular advantages over murine monoclonal antibodies, particularly insofar as they can be used therapeutically in humans. Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the present invention. Thus, the invention contemplates, in one embodiment, a monoclonal antibody of this invention that is humanized by grafting to introduce components of the human immune system without substantially interfering with the ability of the antibody to bind antigen.

The use of a molecular cloning approach to generate antibodies, particularly monoclonal antibodies, and more particularly single chain monoclonal antibodies, is also contemplated. The production of single chain antibodies has been described in the art, see e.g., U.S. Pat. No. 5,260,203, the contents of which are herein incorporated by reference. For this, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by H and L chain combination in a single chain, which further increases the chance of finding appropriate antibodies. Thus, an antibody of the present invention, or a “derivative” of an antibody of the present invention pertains to a single polypeptide chain binding molecule which has binding specificity and affinity substantially similar to the binding specificity and affinity of the light and heavy chain aggregate variable region of an antibody described herein.

Other Modulators

Given the disclosure of the CYP51 biological activity herein, it is also contemplated that other chemical compounds may be used to modulate CYP51 activity, and particularly MT CYP51 biological activity, in accordance with the methods of the present invention. The identification of such compounds is facilitated by the description of screening assays directed to MT CYP51 activity presented above and in view of the highly conserved nature of biologically active CYP51 polypeptides in plants, animals, fungi and bacteria, as described hereinabove.

### EXAMPLES

The following Examples have been included to illustrate preferred modes of the invention. Certain aspects of the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Overview of Examples

Sterol 14α-demethylase encoded by CYP51 is a mixed-function oxidase involved in sterol synthesis in eukaryotic organisms. Using genomic DNA from mycobacterial strain H₃₇Rv, applicants have unambiguously established that the MT CYP51-like gene encodes a bacterial sterol 14α-demethylase. Expression of the Mycobacterium tuberculosis CYP51 gene in Escherichia coli yields a P450 which when purified to homogeneity has a molecular weight of about 50 kD on SDS-PAGE, and binds both sterol substrates and azole inhibitors of P450 14α-demethylases. It catalyzes 14α-demethylation of lanosterol, 24,25-dihydrolanosterol (DHL) and obtusifoliol to produce the 8,14-dienes stereoselectively as shown by GC/MS and ¹HNMR analysis. Both flavodoxin and ferredoxin redox systems are able to support this enzymatic activity. Structural requirements of a 14α-methyl group and Δ⁸⁽⁹⁾-bond were established by comparing binding of pairs of sterol substrate that differed in a single molecular feature, e.g., cycloartenol paired with lanosterol. These substrate requirements are similar to those established for plant and animal P450 14α-demethylases.

### General Experimental Methods and Materials Used in Examples

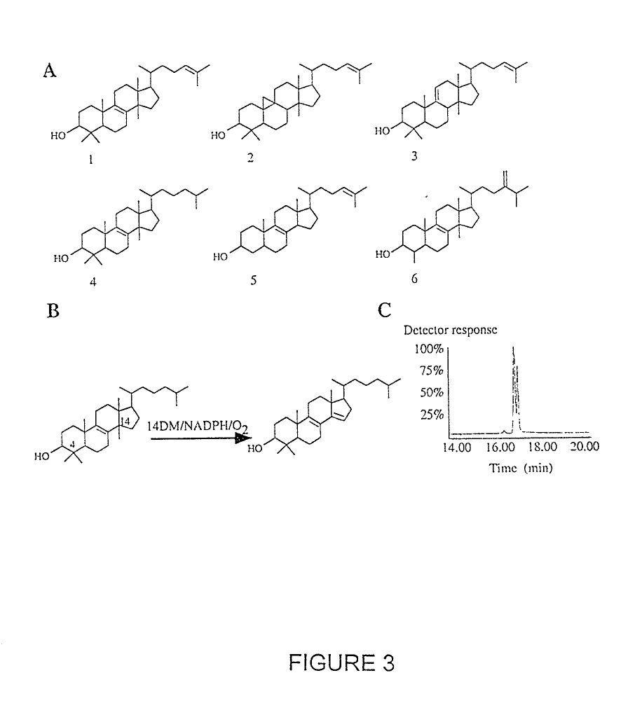

Absolute spectra of purified MT P45014DM were recorded as described by Sato and Omura (1964) J. Biol. Chem. 239:2379-85. Protein quantification was performed using the Bradford method. DNA sequencing was carried out using automated (ABI PRISM™ Dye Terminator Cycle Sequencing Ready Reaction kit and ABI PRISM™ 377 DNA sequencer) and manual sequencing (SequiTherm™ Cycle Sequencing, Epicentre Technologies, Madison, Wis.). Sterols were obtained from the Nes collection (Venkatramesh et al., (1996) Biochim. Biophys. Acta 1299:313-24; Nes, W. D., et al. (1993) Arch. Biochem. Biophys. 300:724-33) and purified by HPLC before assay with P45014DM. The six sterols assayed with MT P45014DM are shown in FIG. 3A.

### Example 1

### Isolation of MT CYP51 Gene and Gene Product

A preferred embodiment of the CYP51 gene of the present invention was isolated from Mycobacterium tuberculosis (MT). As such, the experiments disclosed in this Example describe the first characterization of a CYP51 gene encoding the cytochrome P450 enzyme CYP450 14α-demethylase (P45014DM) in bacteria. Indeed, from the combination of results, the interrelationships of substrate functional groups within the active site show that oxidative portions of the sterol biosynthetic pathway are present in procaryotes. The isolated and characterized amino acid sequence possessed about a 35% amino acid identity with CYP51 in animals, plants and fungi.

The MT CYP51 gene was cloned from MT DNA using PCR technology and was then inserted into a bacterial expression vector according to standard techniques as are described herein. The cloning protocol also added four histidine residues at the carboxy terminus of the protein (as set forth in SEQ ID NO's: 1 and 2) for use in purification of the expressed protein by Ni+² affinity chromatography. The bacterial expression vector was transfected into bacteria and the bacteria were cultured according to techniques as described herein. The expressed protein was then purified by Ni⁺² affinity chromatography.

The spectral properties of the expressed protein clearly demonstrated that it was a CYP450 enzyme. Optical methods demonstrated that the protein binds lanosterol, dihydrolanosterol and obtusifoliol, which are known to be substrates of different forms of CYP450 14DM. Notably, the isolated recombinant MT CYP51 protein did not bind 24-methylene-dihydrolanosterol, which is a substrate for fungal CYP45014DM.

Genomic DNA from MT strain H₃₇Rv was provided by the TB Research Materials and Vaccine Testing Contract (NO1 Al-75320) at Colorado State University and the MT CYP51-like gene was cloned by PCR using Vent polymerase (Biolabs, Inc., Bountiful, Utah).

Primers were designed based on the sequence of cosmid MTCY369 from the MT genome, the upstream primer 5′-cgccatatgagcgctgttgcactaccc-3′ (SEQ ID NO:11) except the first 6 bases being complementary to the sequence between bases 7495-7475 which is predicted to encode the N-terminal sequence of the MT CYP51-like protein. The downstream primer 5′-cgcaagcttcagtgatggtgatgaactcccgttcgccggcggtagc-3′ (SEQ ID NO:12), from bases 24 to 46, is identical to MTCY369 sequence between bases 6143-6165.

For the Fdx gene, the upstream primer 5′-cgccatatgggctatcgagtcgaagcc-3′ (SEQ ID NO:13) except the first 6 bases, is complementary to MTCY369 sequence between bases 6137-6117, and the downstream primer 5′-cgcaagcttcagtgatggtgatgctctccc gtttctcggatggacagtgcctggg-3′ (SEQ ID NO:14) from bases 24 to 55 is identical to bases 5934-5965. The stop codon was removed in each gene and 4 histidine codons followed by a new stop codon (bold characters) inserted in the 3′-end of the coding sequences. The underlined bases are Ndel cloning sites including the initiator codon in the upstream primers and HindIII cloning sites in the downstream primers.

Amplification conditions were 94° C. for 5 min then 30 cycles of 94° C. for 30sec, 50° C. for 30 sec and 72° C. for 45 sec. The PCR program ended using one polymerization step at 72° C. for 10 min and the product was separated by electrophoresis on a 1% agarose gel. Bands of the expected sizes of MT P45014DM (1377 bp) and Fdx (233 bp) were eluted from the gel using Quigen II kit (Quigen, Inc., Chatsworth, Calif.).

Following digestion by Ndel and HindIII, the cDNAs were cloned into the E. coli expression vector pet17b (Novagen, Madison, Wis.) giving MTP450/pet17b and MTFdx/pet17b. Those vectors were transformed separately into competent HMS174 (Novagen, Madison, Wis.) cells. Single ampicillin-resistant colonies from each transformant were grown overnight at 37° C. in 5 ml of Terrific Broth containing 100 μg/ml of ampicillin. These precultures were used to inoculate (1:100) 500 ml of modified Terrific Broth medium (100 μg/ml of ampicillin) (O'Keefe, D. P., et al. (1991) Biochemistry 30:447-455). After 5 hour (h) growth at 37° C. in a shaking incubator at 240 rpm, the culture was induced using 1 mM isopropyl β-D-thiogalactopyranoside (Calbiochem, San Diego, Calif.). At the same time, 67-aminolevulinic acid (Sigma, St. Louis, Mo.) was added to 2 mM final concentration for P450 expression. Growth was continued at 30° C. with shaking at 190 rpm for 20 hours.

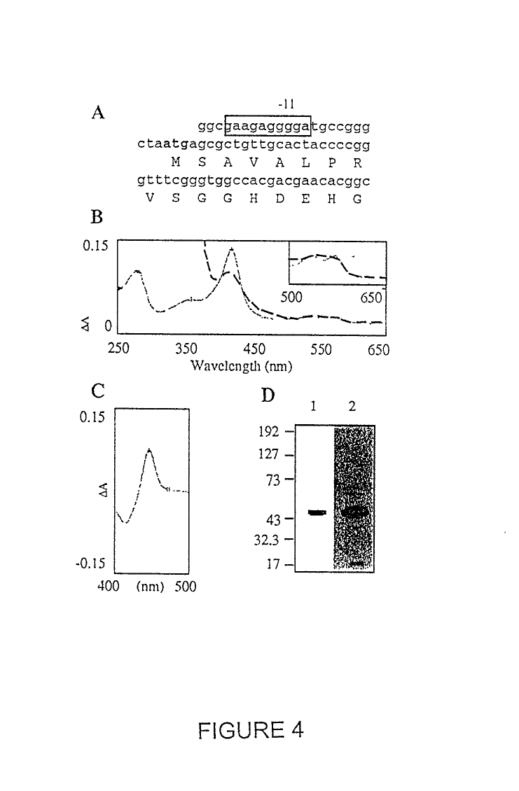

A purine rich region, GAAGAGGGGA, located 10 bp upstream from the start codon is a potential Shine-Dalgarno sequence (FIG. 4A), the length of the spacer between this and the start codon (10 bp) being similar to other mycobacterial genes (Dale, J. W. & Patki, A. (1990) in Molecular Biology of the Mycobacteria, ed. McFadden, J. (Academic Press, San Diego, Calif.), pp. 173-198). MT CYP51 produced in E. coli (2.5 μmol/L) has a typical P450 reduced-CO spectrum (FIG. 4C) as observed by Aoyama, Y., et al., (1998) J. Biochem. (Tokyo) 124:694-696. Cell fractionation reveals that MT P45014DM is soluble, no P450 being detected in the membranes as seen upon expression in JM109 E. coli strain in accordance with techniques described by Aoyama, Y., et al., (1998) J. Biochem. (Tokyo) 124:694-696.

### Example 2

### MT P45014DM Purification.

Three liters (L) of MT P450 culture were pelleted and re-suspended in 200 ml of TES buffer in accordance with techniques described by Jenkins, C. M. & Waterman, M. R. (1994) J. Biol. Chem. 269: 27401-8. Following addition of lysozyme (0.5 mg/ml) and stirring at 4° C. for 15 min, one volume of ice-cold water containing 0.1 mM EDTA was slowly added and stirring continued for 30 min. Spheroplasts were pelleted at 3,000 g for 15 min. The supernatant (fraction A) was centrifuged at 225,000 g for 30 min following addition of DNAase I (1 μg/ml) and stirring at 4° C. for 15 min. Spheroplasts were resuspended in 50 ml of 2 fold diluted TES buffer, sonicated using a Branson sonifier (Model 250) at duty cycle 30-40, 50% maximal output for 30 sec at room temperature followed by 1 min incubation on ice, repeated 10 times.

Following centrifugation at 225,000 g for 30 min, the supernatant (fraction B) was combined with fraction A and the P450 isolated using a Ni²⁺NTA affinity column (Qiagen, Valencia, Calif.) equilibrated with 50 mM potassium phosphate pH 7.4 and 20% glycerol. After washing with the same buffer containing 50 mM glycine and 500 mM NaCl, the P450 was eluted using 40 mM L-histidine in place of glycine. The P450 eluate was dialyzed overnight against 50 mM potassium phosphate pH 7.4 and 20% glycerol.

One L of MT Fdx culture grown as for MT P450 was pelleted and resuspended in 50 mL of 50 mM potassium phosphate pH 7.4, 0.1 mM EDTA and 20% glycerol. Following addition of lysozyme (0.5 mg/ml) and stirring at 4° C. for 15 min, cells were sonicated as above. The cytosolic fraction after centrifugation at 225,000 g was loaded on a Ni²⁺NTA affinity column. Washing and elution conditions were the same as for MT P450.

After two consecutive Ni²⁺affinity column purification steps, the specific content of the MT P45014DM is about 18 nmol/mg and a single band is observed on SDS-PAGE at about 50 kD, the predicted molecular weight from the sequence being 51.4 kD (FIG. 4D). The oxidized absolute spectrum of the purified enzyme, in the absence of substrate, showed a Soret band at 417 nm and α-, β- and δ-bands at 569, 535 and 369 nm, typical for low spin cytochrome P450 (FIG. 4B). Reduction by sodium hydrosulfite results in a Soret peak at411 nm.

### Example 3

### Antibody Production

Polyclonal antibodies against MT P45014DM purified by two passes over Ni²⁺NTA were raised in white New Zealand rabbits, injected with 0.5 mg MT P45014DM mixed with either complete Freund's adjuvant (Sigma, St. Louis, Mo.) or TiterMax@Gold (Cytrx Corp., Norcross, Ga.). Two weeks later, the rabbit injected with Freund's adjuvant was boosted using 0.5 mg MT P45014DM in Freund's incomplete adjuvant (Sigma, St. Louis, Mo.) and the antiserum was collected after 4 weeks. From the rabbit injected with TiterMax@Gold™, antiserum was collected after 19 days.

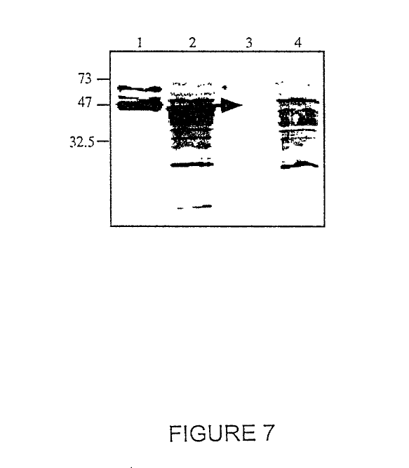

Immunoblot analysis was carried out on the cytosolic fraction of the MT virulent strain H₃₇Rv (FIG. 7). A band near the expected molecular weight but lower than for purified recombinant MT P45014DM was obtained. Using antiserum depleted with an excess of purified recombinant MT P45014DM, this cytosolic band as well as that for purified P45014DM was dramatically reduced. This indicates that the MT P45014DM is expressed in MT.

The difference in size between the recombinant and MT proteins might be explained by the presence of four additional histidines at the C-terminus of the recombinant enzyme. Such modification can affect the mobility of proteins during SDS-PAGE. In fact, a single amino acid mutation in P4501 B1 expressed in E. coli results in shift to a greater size on the SDS-PAGE, as described by Shimada, T., et al. (1998) Arch. Biochem. Biophys. 357:111-120.

### Example 4

### Characterization of Enzymatic Activity of MT CYP 51 Protein

CYP450 enzymes do not function as independent proteins. Rather, they require a reductase system to support their enzymatic activity. In eukaryotes CYP450 activity in the endoplasmic reticulum (ER), is supported by the ER protein NADPH cytochrome P450 reductase. CYP45014DM is an ER protein in animals, plants and fungi and thus, its activity is supported by CYP450 reductase.

In most bacterial CYP450s, enzymatic activities are supported by a two component reductase system, a ferredoxin which interacts with the CYP450 and a ferredoxin reductase. With respect to the MT CYP51 gene product of the present invention, the ability of eukaryotic reductase systems, including rat CYP450 reductase and bovine adrenodoxin and adrenodoxin reductase (a mammalian ferredoxin/ferredixin reductase system), were tested for the ability to reduce MT P45014DM. Neither reduced the enzyme. Applicants then used a two-component system of flavodoxin/flavodoxin reductase, which applicants previously noted in E. coli and other bacteria, to evaluate whether this reductase system would support the activity of the MT P45014DM of the present invention. Jenkins and Waterman, J. Biol. Chem. 264:27401-27408 (1994). This system was observed to readily support 14α-demethylase activity with respect to dihydrolanosterol and obtusifoliol.

Materials and Methods. Activities of P450 enzymes require support of a reductase, and a functional reductase system for MT P450s was unknown. The capacity of rat microsomal NADPH cytochrome P450 reductase, of bovine adrenodoxin/adrenodoxin reductase and E. coli Fld/flavodoxin reductase (Fdr) (Jenkins, C. M. & Waterman, M. R. (1994) J. Biol. Chem. 269:27401-8) to reduce MT P45014DM was determined by formation of the reduced-CO spectrum.

MT P45014DM (200 pmol) and rat P450 reductase (200 pmol) were incubated in 10 mM potassium phosphate buffer pH 7.4 containing 20% glycerol and 200 μM final concentration of lanosterol with or without 100 μg/ml sonicated dilauroyl-L-a-phosphatidylcholine. After several cycles of degassing and bubbling with carbon monoxide, NADPH (Calbiochem, San Diego, Calif.) was added (final concentration 1 mM) and the reduced-CO spectrum recorded. In the control experiment, 40 μM final concentration of progesterone was added to 200 pmol bovine 17α-hydroxylase P450.

To study the bovine adrenodoxin/adrenoxin reductase system, 20 mM Tris-HCl pH 7.4 buffer containing 0.2% Tween 20, 4 mM MgCl₂ and 200 μM lanosterol (final concentrations) was used with MT P45014DM (100 pmol)/adrenodoxin (1 nmol)/adrenodoxin reductase (100 pmol) with 1 mM NADPH. Bovine cholesterol side chain cleavage P450 (100 pmol) with the substrate 25-hydroxycholesterol (30 μM final concentration) was used as positive control. Using the Fld/Fdr system, 500 pmol of MT P45014DM, 2.5 nmol of Fld and 500 pmol of Fdr were incubated on ice (10 min) in 3-(N-morpholino)propane sulfonic acid buffer containing 200 μM final concentration of lanosterol and 1 mM NADPH with or without 100 μg/ml dilauroyl-L-a-phosphatidylcholine. In the control experiment, 40 mM of progesterone was added to 500 pmol P450 17α-hydroxylase.

It was observed that despite the fact that lanosterol bound the MT CYP51 protein of the present invention, lanosterol was not a substrate of the enzyme. No enzymatic activity was observed with respect to lanosterol. But, using high performance liquid chromatography (HPLC), mass spectroscopy (MS) and nuclear magnetic resonance (NMR) it was established that the P45014DM protein of the present invention catalyzed 14α-demethylase activity.

To investigate MT P45014DM enzymatic activity, it was first necessary to determine which electron donor can reduce the hemoprotein. In eukaryotes, P450s are localized in either the endoplasmic reticulum and reduced by ubiquitous NADPH cytochrome P450 reductase (Vermilion, J. L. & Coon, M. J. (1978) J. Biol. Chem. 253:8812-9) or in the inner mitochondrial membrane and reduced by a 2 component system of a flavoprotein reductase and a 2Fe-2S protein (Coghlan, V. M. & Vickery L. E. (1991) J. Biol. Chem. 266:18606-12). Neither reducing system was capable of reducing MT P45014DM (Table 2). Nonetheless, applicants found that it can be reduced by a 2 component system, Fld/Fdr from E. coli. Fld/Fdr reduces MT P45014DM at about 20% of the full reduction by sodium hydrosulfite when using the P450:Fld:Fdr ratio of 1:5:1 which reduces bovine P450c17 at 77% (Table 2).

TABLE 2
MT P45014DM Reduction By Different Etectron Donors

| | | rat P450 | | | |
|---|---|---|---|---|---|
| Adx/Adr | | reductase | | Fld/Fdr | |
| SCC | MT | bC17 | MT | bC17 | MT |
| 80% | n.r | 73% | n.r. | 77% | 20% |

CO-based reduction of purified recombinant P450s by adrenodoxin/adrenodoxin reductase, rat P450 reductase and E. coli Fld/Fdr. Reduction is compared to 100% determined by sodium hydrosulfite. Adx/Adr is adrenodoxin/adrenodoxin reductase, SCC is the cholesterol side cleavage P450, bC17 is bovine 17α-hydroxylase and MT is the mycobacterial 14α-demethylase. n.r.=no reduction.

### Example 5

### Reconstituted Catalytic Activity and Sterol Analysis

MT P45014DM (365 pmol) was incubated on ice (10 min) with 18 nmol Fld and 2 nmol of Fdr or 18 nmol MT Fdx and 2 nmol spinach ferredoxin reductase (Fnr). Since the electron donor to MT Fdx is unknown, Fnr (Sigma, St. Louis, Mo.) shown to reduce ferredoxins from S. griseolus (Bauer, S. & Shiloach, J. (1974) Biotechnol. Bioeng. 16:933-41), was used. Substrate dispersed in Triton WR 1339 was resuspended in 3-(N-morpholino) propane sulfonic acid buffer (Stromstedt, M. et al. (1996) Arch. Biochem.Biophys. 329:73-81). After mixing, the reaction was initiated (2 mM NADPH) in a final volume of 500 μl. Following catalysis, sterols were extracted twice using 5 volumes of ethyl acetate for small scale reactions or hexane for large scale experiments. In the latter case, 1 volume of methanol containing 10% KOH was used to stop the reaction. One volume of dimethyl sulfoxide was then added and after heating at 90° C. and cooling to room temperature, sterols extracted 3 times using 3 vol of hexane and evaporated to dryness.

Radiolabeled dihydrolanosterol ([24-³H]DHL) (Fischer, R. T., et al. (1989) J. Lipid Res. 30:1621-32) and its tritiated 14-desmethyl sterol product were separated by HPLC on a Nova-Pak C₁₈ column (Stromstedt, M. et al. (1996) Arch. Biochem. Biophys. 329:73-81). Nonradioactive sterols were separated by HPLC on a 25 cm Zorbax C₁₈ column (Dupont, Boston, Mass.; 5 mm particle size 4.6 mm i.d) by elution with 100% methanol at room temperature (flow rate of 1 ml/min). Thin layer chromatography was performed on 250μsilica gel G plates, developed twice with benzene/ether (85/15).

GLC analysis was performed on a three-foot spiral 3% SE-30 packed column operated isothermally at 245° C. GLC-MS was performed on a Hewlett Packard 5973 Mass Selective Detector interfaced with a 6890 GC system. The capillary column for GLC was a 30-m DB-5 column 250 μM×0.25 μM (from J&W Scientific, Folsom, Calif.). The temperature program was operated at: 170° C. hold for 1 min; ramp at 20° C./min to 280° C.; hold for 15 min. Mass spectroscopy (MS) was performed using MS transfer line at 280° C., with the inlet injector port kept at 250° C. The MS ion source temperature was maintained at 230° C. Helium gas, used as carrier, was maintained at a flow rate of 1.2 ml/min. Proton nuclear magnetic resonance (¹HNMR) spectroscopy was performed on samples dissolved in deuteriated chloroform at ambient temperature using a AF-300 spectrometer (Billeria, Mass.) with tetramethylsilane as internal standard in accordance with techniques described by Venkatramesh et al., (1996) Biochim. Biophys. Acta 1299:313-24; Nes, W. D., et al. (1993) Arch. Biochem. Biophys. 300:724-33; Xu, S. H., et al. (1988) J. Chromatogr. 452:377-98; and Nes, W. D., et al. (1998) J. Amer. Chem. Soc. 120:5970-5980.

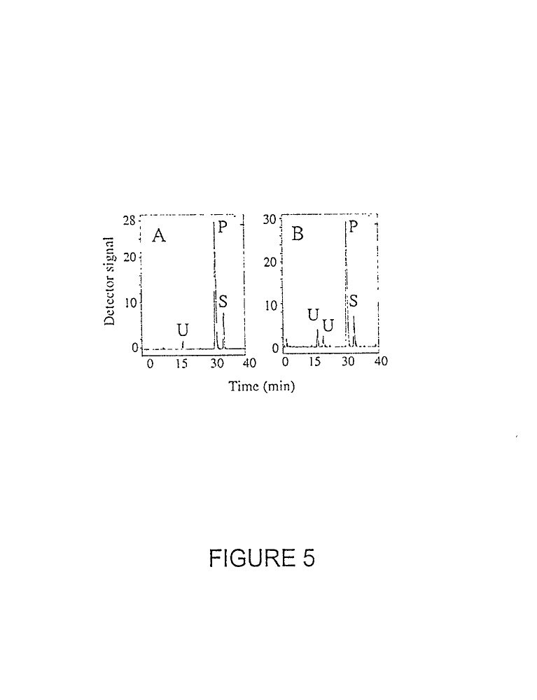

Preliminary studies on metabolism of [24-³H]DHL by MT P45014DM suggested the reconstituted enzyme using Fld/Fdr catalyzed 14α-demethylation. Cloning Fdx from MT showed that this 3Fe-4S ferredoxin was able to support the MT P45014DM activity to a similar level as E. coli Fld/Fdr when spinach Fnr was used as a Fdx electron donor (FIG. 5). In order to characterize the product, scaled-up batch enzyme experiments using Fld/Fdr as electron donor and three different nonradioactive sterol substrates lanosterol, DHL and obtusifoliol were carried out overnight with 50 μM sterol and 365 pmol enzyme per assay. Total product recovered from the quenched reaction mixtures was 1% for lanosterol, 20% for DHL (FIG. 5C) and 98% for obtusifoliol. From incubation with lanosterol, a single sterol product was detected on TLC at R_{f} 0.5 (the R_{f} value distinguishes whether C-demethylation occurs at C4, R_{f} 0.43, or C14, R_{f} 0.50), by GC (3% Se-30: retention time relative to cholesterol-RRTc, 1.62) and MS (M⁺, 410 and related diagnostic ions at m/z, 395, 392, 377, 357, 328 ) and UV (in ethanol) at λₘₐₓ 248 nm for a 8,14-diene (Goad, L. J. & Akhisa, T. (1997) Analysis of Sterols (Blakie, N.Y.).

From incubation with DHL, a single sterol product was detected and identified: TLC, R_{f} 0.5; GLC, RRTc 1.53; MS, M⁺412 (and related ions at 397, 394, 279, 351, 312, 285, 266, 245, 227, 159); UV (in ethanol) λₘₐₓ 248 nm; ¹H NMR analysis of the sample exhibited 4 singlets and 3 doublets in the methyl region of the spectrum between δ0.76 and 1.01 ppm consistent with loss of a methyl group from C14 and a single chemical shift at δ5.34 ppm in the olefinic region corresponding to the Δ¹⁴⁽¹⁵⁾-bond. These structural assignments indicate a 4,4-dimethyl Δ^{8,14(15)}-sterol (Nes, W. D., et al. (1993) Arch. Biochem. Biophys. 300:724-33). From incubation with obtusifoliol, a single sterol product was detected and identified: TLC, R_{f} 0.43 (characteristic migration on TLC for a C4-monomethyl sterol), GLC, RRTc 1.55; MS, M⁺410 (and related ions at m/z 395, 392, 379, 357, 328, 267, 247, 227, 189) UV (in ethanol) λₘₐₓ 248 nm.

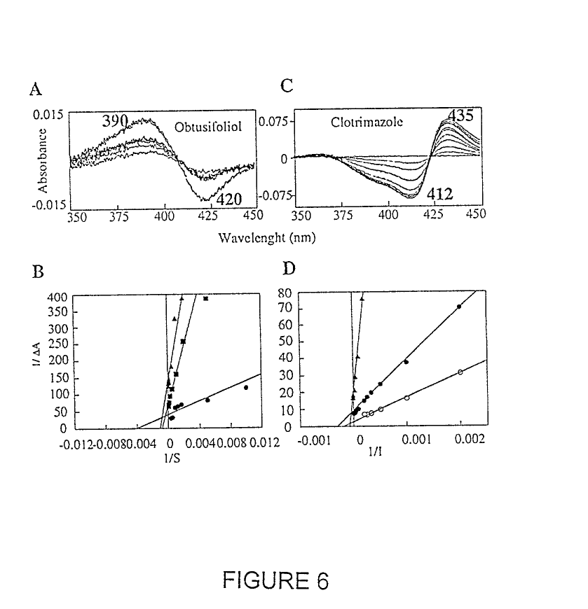

Substrate binding. In the absence of substrates, most P450 enzymes are low spin (Guengerich, F. P. (1983) Biochemistry 22:2811-20). Substrate addition shifts the heme to the high spin state. For MT P45014DM as for most P450s, the change in spin state leads to a peak at 390 nm and a trough at 420 nm in the substrate induced difference spectrum (FIG. 6A). The amplitude of this difference is proportional to the P450-substrate complex. Addition of increasing amounts of substrate permits estimation of a binding constant similar to the K₅ value. Binding constants were determined for obtusifoliol, lanosterol and DHL. Obtusifoliol binds to the enzyme with a K_{S} value of 350±150 nM whereas DHL and lanosterol bind to the enzyme less effectively, ca. 1±0.5 μM each (FIG. 6B). Neither parkeol, cycloartenol, nor zymosterol (FIG. 3A) were found to bind to the enzyme.

Azole binding spectra. Binding of ketoconazole, clotrimazole and fluconazole, known for their ability to inhibit 14α-demethylase activities (Yoshida, Y. & Aoyama, Y. (1987) Biochem. Pharmacol. 36:229-35; Salmon, F., et al. (1992) Arch. Biochem. Biophys. 297:123-31) was examined for MT P45014DM. These molecules produce type II binding spectra due to binding of the azole nitrogen to the 6th coordination position of the heme iron. The type II binding spectrum is characterized by a peak at 434 and a trough at 412 nm (FIG. 6C). Similar to the type I spectra, the P450-inhibitor complex can be titrated leading to an estimation of the inhibitor Ks (FIG. 6D). For ketoconazole and clotrimazole, these values are around 5 μM whereas for fluconazole around 10 μM. Ketoconazole (20 μM) was found to inhibit the 14α-demethylation of DHL by MT P45014DM.

### Example 6

### Crystalline Structure of MT CYP51 Enzyme

This example describes the characterization of the three-dimensional (3-D) crystalline structure of the MT CYP51 gene product of the present invention. It is desirable to determine the 3-D structure of this protein at high resolution because of its activity as a 14α-demethylase enzyme. Other known forms of CYP45014DM's are from eukaryotes and are integral membrane proteins in the ER. Efforts to crystalize membrane bound proteins are difficult at best and only a small number of structures of these proteins are known. No crystalline structure is currently available for a eukaryotic CYP45014DM.

However, bacterial P450s are soluble proteins and are much easier to crystalize. At least six 3-D structures of bacterial CYP450s have been solved at high resolution using x-ray crystallography. Since the MT P45014DM protein of the present invention is a soluble protein, the crystalline structure of this 14α-demethylase is much easier to solve as compared to eukaryotic CYP45014DM's and thus, such a crystalline form is contemplated in accordance with the present invention. Indeed, obtaining crystals of quality sufficient for determining the structure of a CYP45014DM enzyme has not been achievable until the crystallization of MT CYP45014DM as disclosed herein. Thus, the crystalline structure of MT CYP45014DM is used to model the tertiary structure of related proteins in accordance with art-recognized techniques, such as those used in modeling the structure of renin using the tertiary structure of endothiapepsin as a starting point for the derivation (Blundell et al. (1983) Nature 304:273-275). Additional crystallization techniques are described in U.S. Pat. Nos. 5,322,933; 5,834,228; and 5,872,011, the entire contents of which are herein incorporated by reference.

Furthermore, current methods of tertiary structure determination that do not rely on X-ray diffraction techniques and thus do not require crystallization of the protein, such as NMR techniques, are made much simpler if a model of the structure is available for refinement using the additional data gathered by the alternative technique. The elucidation of the tertiary crystalline structure of MT CYP45014DM in accordance with the present invention thus provides a starting point for investigation into structure of all CYP450 enzymes including particularly, but not limited to, the various species of CYP45014DM's.

By way of particular example, CYP450 14α-demethylase in fungi (yeast) is targeted by drugs used for the treatment of yeast infections including jock itch and athlete's foot by topical treatment. Inhibitors of P45014DM such as ketoconazole bind tightly to the yeast enzyme in the active site, thereby preventing ergosterol biosynthesis and killing the yeast. Thus, the elucidation of the structure of the P45014DM active site in accordance with this example facilitates the design of even more effective drugs.

Additionally, the development of very specific drugs for the treatment of Candida albicans infections in immunocompromised (i.e. HIV) individuals is also facilitated. This infection, which can be deadly, requires systemic treatment with azole inhibitors such as ketoconazole. But, these inhibitors, when used systemically, will also inhibit the function of endogenous CYP450s in the human host. For example, inhibitors of Candida CYP45014DM will also inhibit human CYP45014DM and probably many other human CYP450s. Therefore systemic use of CYP450 inhibitors is limited to only those patients in dire need of treatment. Accordingly, the resolution of the 3-D structure of MT CYP45014DM in accordance with this example provides a very useful tool in rational drug design for more specific inhibitors of yeast, MT, and other CYP45014DMs.

### Example 7

### Methods of Treating Tuberculosis

The characterization of the CYP51 enzyme of the present invention in MT leads to the analysis of the function of this enzyme in the disease tuberculosis and of whether this enzyme represents a new drug target for the treatment of tuberculosis. Particularly, the effects of three azole inhibitors on the growth of MT H37RA, an alternated strain of MTH37R which is the pathogenic strain whose genomic sequence was determined by Cole et al., Nature 193:537-544 (1998) (bacterial strains provided by Dr. Dean Crick at Colorado State University) were examined.

As contemplated by applicants in accordance with the present invention, it was observed that ketoconazole has a profound effect on the growth of MT, stopping growth at about a 25 μM concentration. This result indicates that the MT P45014DM enzyme isolated in accordance with the present invention plays an essential role in MT growth and that azole inhibitors of CYP450 enzymatic activity provide new candidates for drugs in the treatment of tuberculosis. The targeting of ketoconazole to P45014DM in MT also leads to the identification of additional specific azole inhibitors which have far greater specificity for the MT CYP45014DM enzyme as compared to the human CYP45014DM enzyme. Such inhibitors therefore will have fewer side effects than less specific inhibitors. Thus, as described above, a method of screening for highly specific inhibitors of MT CYP45014DM in accordance with the screening methods described hereinabove comprises another aspect of the present invention.

### Example 8

### Modulation of CYP45014DM Biological Activity

Given the demonstrated biological activity of the isolated MT CYP51 enzyme of the present invention as a 14α-demethylase, and given the characterization of the structure of this enzyme as described hereinabove, it is contemplated that a method of screening for specific inhibitors of cholesterol synthesis comprises an additional aspect of the present invention. Screening for a modulator of cholesterol synthesis which preferentially modulates CYP45014DM activity is particularly contemplated. By the term “preferentially” it is meant that the contemplated modulator tends to modulate the activity of CYP45014DM enzymes to a greater extent as compared to other CYP450 enzymes. The identification of such modulators is facilitated by the characterization of the crystalline structure of the MT CYP51 polypeptide of the present invention as well as the rational drug design methods disclosed hereinabove.

A method of modulating cholesterol synthesis comprising administering an effective amount of a cholesterol synthesis modulating composition to a vertebrate subject in need thereof is also contemplated in accordance with the present invention. Preferably, the cholesterol synthesis modulating composition comprises a therapeutically effective amount of a compound which preferentially modulates the activity of a CYP45014DM enzyme in the vertebrate subject.

Additionally, given the importance of cholesterol and steroid synthesis during spermatogenesis and given that the highest level of expression of the CYP45014DM enzyme in mammals, and particularly in humans, is found in developing spermatids during spermatogenesis, a screening method for a therapeutic agent useful in the modulation of spermatogenesis is contemplated in accordance with the present invention. Screening for a modulator of spermatogenesis which preferentially modulates CYP45014DM activity is particularly contemplated. By the term “preferentially” it is meant that the contemplated modulator tends to modulate the activity of CYP45014DM enzymes to a greater extent as compared to other CYP450 enzymes. The identification of such modulators is facilitated by the characterization of the crystalline structure of the MT CYP51 polypeptide of the present invention as well as the rational drug design methods disclosed hereinabove.

A method of modulating spermatogenesis comprising administering an effective amount of a spermatogenesis modulating composition to a vertebrate subject in which such modulation is desirable is also contemplated in accordance with the present invention. Preferably, the spermatogenesis modulating composition comprises a therapeutically effective amount of a compound which preferentially modulates the activity of a CYP45014DM enzyme in the vertebrate subject.

### Discussion of Examples

The results of the Examples demonstrate that MT contains a gene encoding an enzyme that catalyzes removal of the sterol 14α-methyl group stereoselectively, producing the 8,14-diene. The influence of substrate structure on MT P45014DM sterol binding has been determined using a series of substrates that differ in a single molecular feature or in a combination of similar features. The tendency for preferential binding of obtusifoliol compared with the five other sterols tested indicates that the active site accommodates sterol side chains with a C24-alkyl group, suggesting the bacterial enzyme is plant/fungal-like in its active site topology. Obtusifoliol was also found to be the best substrate for the MT P45014DM.

The inability of parkeol or cycloartenol, structural isomers of lanosterol, to bind MT P45014DM indicates that the orientation of the substrate assumed upon binding requires a specific pseudoplanar conformation of the ring system and specific equatorially oriented tilt of the C3-hydroxyl group; analogous structural requirements as observed for the sterol methyl transferase enzyme from fungi and plants (Venkatramesh et al., (1996) Biochim. Biophys. Acta 1299:313-24; Nes, W. D., et al. (1991) J. Biol. Chem. 266:15202-12). The lack of zymosterol binding (4,4,14-tridesmethyl lanosterol) indicates that one or both of the C4- and C14-methyl groups are important in sterol binding.

The Δ⁸-bond is a critical stereoelectronic element of recognition; in each of the three sterols that were found to undergo 14α-demethylation by MT P45014DM the product of the multi-step reaction was a sterol with the conjugated Δ¹⁴⁽¹⁵⁾-bond system, suggesting the bacterial enzyme has evolved to bind and catalyze 14α-methyl sterols in a manner similar to P45014DM enzymes from higher species (Yoshida, Y., et al. (1997) J. Biochem. (Tokyo) 122:1122-8). Clearly, there is a conservation in sterol specificity for the P45014DM enzyme from primitive bacteria to advanced fungal and plant systems. The ketoconazole binding constant estimated for maize microsomes is 10 μM (Salmon, F., et al. (1 992) Arch. Biochem. Biophys. 297:123-31), about the same as that for MT P45014DM emphasizing similarities between bacterial and eukaryote enzymes.

A purine rich region located 10 bp upstream the ATG, is associated with the MT CYP51 gene. Similar sequences are associated with other MT genes such as TB dnaj and TB 65 (Dale, J. W. & Patki, A. (1990) in Molecular Biology of the Mycobacteria, ed. McFadden, J. (Academic Press, San Diego), pp. 173-198). The structure and the location of this putative MT CYP51 Shine-Dalgarno sequence is also in agreement with what is known in the most studied bacterium, E. coli where the purine rich region is separated from the ATG by 5 to 12 bases (De Boer, H. A. & Hui, A. (1991) in Gene Expression Technology, ed. Goeddel, D. V. (Academic Press, Inc, San Diego, Calif.), Vol. 185, pp. 103-114). No such sequence could be identified upstream the Fdx gene in the P450 open reading frame, suggesting that the 2 genes which are separated by only 2 bp might be expressed as a polycistronic RNA.

MT P45014DM is the first endogeneous P450 found to accept electrons from both an iron-sulfur protein (Fdx) and a FMN containing protein (Fld). Perhaps this reflects a transition in the P450 evolution between procaryotic electron transfer (iron-sulfur protein) and the eukaryotic type (FMN containing protein for microsomal P450s). The 3Fe-4S ferredoxin is contemplated as a good candidate for the endogeneous MT reductase.

The formulation of binding topology from studies with sterol substrates and the sensitivity of the P45014DM to azole inhibitors is consistent with MT having a functional sterol pathway, and further refines the general picture of sterol evolution which has emerged from classical natural product chemistry approaches to identify sterol biosynthetic pathways. The identification of P45014DM in MT and its contemplated role in sterol biosynthesis supports the recent demonstration that cholesterol biosynthesis occurs in M. smegmatis via a mevalonic pathway (Lamb, D. C., et al. (1998) FEBS Lett. 437:142-144). Targeting MT P45014DM provides new options in drug design for new treatments of tuberculosis, a disease infecting one third of the world population (Fenton, M. J. & Vermeulen M. W. (1996) Infect. Immun. 64:683).

### References

The publications and other materials used herein to illuminate the background of the invention, and in particular cases, to provide additional details respecting the practice, are incorporated herein by reference, and for convenience, are referenced by author and date in the foregoing text, and respectively grouped in the list of references presented below.

Adelman et al. (1983) DNA 2:183.

Aoyama, Y., et al., (1998) J. Biochem. (Tokyo) 124:694-696.

Ausubel et al. (1992) Current Protocols in Molecular Biology, (J. Wylie & Sons, N.Y.)

Bassing et al., Science 263:87-89 (1994).

Bauer, S. & Shiloach, J. (1974) Biotechnol Bioeng. 16:933-41.

Bird, C. W., Lynch, J. M., Pirt, F. J. & Reid, W. W. (1971) Nature 230:473-4.

Blundell et al., Nature 304:273-275 (1983).

Bodanszky et al., “Peptide Synthesis”, John Wiley & Sons, 2^{nd} Ed. (1976).

Burrow, W., Moudler, J. W. & Lewert, R. M. (1 966) Text book of Microbiology (18th Edition. Sanders, Philadelphia) p. 748.

Chien et al., Proc. Natl. Acad. Sci. USA 88:9578-9582 (1991).

Clapham et al., Nature 365:403-406 (1993).

Coghlan, V. M. & Vickery L. E. (1991) J. Biol. Chem. 266:18606-12

Cole, S. T., et al., (1998) Nature 393:537-44.

Crea et al. (1978) Proc. Natl. Acad. Sci. U.S.A, 75:5765.

Dale, J. W. & Patki, A. (1990) in Molecular Biology of the Mycobacteria, ed.

McFadden, J. (Academic Press, San Diego), pp.173-198.

De Boer, H. A. & Hui, A. (1991) in Gene Expression Technology, ed. Goeddel,

D. V. (Academic Press, Inc, San Diego, Calif.), Vol. 185, pp. 103-114.

Durfee et al., Genes Dev. 7:555-569 (1993)

Eichenlaub et al. R. J. Bacteriol 138:559-566, 1979.

Fenton, M. J. & Vermeulen M. W. (1996) Infect. Immun. 64:683.

Fields et al., Int. J. Peptide Protein Res., 35:161-214, 1990

Fischer, R. T., et al. (1989) J. Lipid Res. 30:1621-32.

Gillmor et al. (1997) Nature Struct. Biol. 4:1003-1009.

Goad, L. J. & Akhisa, T. (1997) Analysis of Sterols (Blakie, N.Y.).

Gough, N. M. & Adams, J. M. (1978) Biochemistry 17:5560-6.

Green et al. (1989) J. Invest. Dermatol. 93:486-491.

Gribskov et al. (1986) Nucl. Acids. Res. 14:6745.

Guengerich, F. P. (1983) Biochemistry 22:2811-20.

Harper et al., Cell 75: 805-816 (1993).

Hayashi et al. Cell 89:1165-1173 (1997).

Heldin et al. Nature 390:465-471 (1997).

Hoodless et al., Cell 85:489-500 (1996).

Howell et al. Antibodies: A Laboratory Manual, (Cold Spring Harbor Laboratories)(1988).

Imamura et al. Nature 389:622-626 (1997).

Jenkins, C. M. & Waterman, M. R. (1994) J. Biol. Chem. 269:27401-8.

Keski-Oja et al., J. Cell Biochem. 33:95 (1987).

Kohl, W., Gloe, A. & Reichembach, H. (1983) J. Gen. Microbiol. 129:1629-636.

Kretzschmar et al., Genes Dev. 11:984-995 (1997).

Kyte et al. (1982) J. Mol. Biol. 157: 105.

Lam et al., J. Biol Chem. 270:26511-26522 (1995).

Lamb, D. C., et al. (1998) FEBS Lett. 437:142-144.

Lois. L. M., Campos, N., Surya, R. P., Danielsen, K., Rohmer, M. & Boronat, A. (1998) Proc. Natl. Acad. Sci. USA 95,2105-2110.

Marshall, C. J. Cell 80:179-185 (1995).

McOmie, “Protective Groups in Organic Chemistry”, Plenum Press, New York, (1973).

Meienhofer, “Hormonal Proteins and Peptides”, Vol.2, p.46, Academic Press, New York (1983).

Merrifield, Adv Enzymol, 32:221-96,1969

Messing et al. (1981) Third Cleveland Symposium on Macromolecules and Recombinant DNA, Ed. A. Walton, (Elsevier, Amsterdam).

Nakao et al., Nature 389:631-635 (1997).

Needleman et al., J. Mol. Biol., 48:443 (1970).

Neer et al., Nature 371:297-300 (1994).

Nelson, Methods in Molecular Biology 107:15-24 (1998).

Nes, W. D., et al. (1990) Proc. Natl. Acad. Sci. USA 87:7565-7569.

Nes, W. R. & Nes, W. D. (1980) Lipids In Evolution (Plenum, New York).

Nes, W. D. (1990) Rec. Adv. Phytochem. 24:283-327.

Nes, W. D. (1987) ACS Symp. Ser. 325:304-328.

Nes, W. R. (1974) Lipids 9:596-612.

Nes, W. D., et al. (1993) Arch. Biochem. Biophys. 300:724-33.

Nes, W. D., et al. (1991) J. Biol. Chem. 266:15202-12.

Nes, W. D., et al. (1998) J. Amer. Chem. Soc. 120:5970-5980.

O'Keefe, D. P., et al. (1991) Biochemistry 30:447-455.

Omura, T. & Sato, R. (1964) J. Biol. Chem. 239:2379-85.

Ourisson, G., Rohmer, M. & Poralla, K. (1987) in Annu. Rev. Microbiol. ed.

Ornston, L. N. (Annual Reviews Inc., Calif.) Vol. 42, pp. 301-333.

Pascal, R. A., et al. (1980) J. Amer. Chem. Soc. 102:6599-6601.

PCT Publication WO 93/25521, published Dec. 23, 1993

Philipp, W., et al. Proc. Natl. Acad. Sci. U.S.A. 93 (7): 3132-3137 (1996)

Pinto, W. J., et al. (1983) Biochem. Biophys. Res. Commun. 112:47-54.

Rahier, A. & Taton, M. (1990) Biochem. Soc. Trans. 18:52-6.

Ramgopal, M. & Bloch, K. (1983) Proc. Natl. Acad. Sci. USA 80:712-5.

Salmon, F., et al. (1992) Arch. Biochem. Biophys. 297:123-31.

Sambrook et al. (1989) Molecular Cloning: A Laboratory Manual (Cold Spring Harbor Laboratory Press, Cold Spring Harbor, N.Y.).

Schroder et al., “The Peptides”, Vol. 1, Academic Press (New York) (1965).

Schwartz et al., eds. (1979) Atlas of Protein Sequence and Structure, National Biomedical Research Foundation, pp. 357-358.

Shimada, T., et al. (1998) Arch. Biochem. Biophys. 357:111-120.

Smith et al., Adv. Appl. Math. 2:482 (1981).

Standaert et al., Nature 346:671-674 (1990).

Steward et al., “Solid Phase Peptide Synthesis”, W. H. Freeman Co., San Francisco (1969).

Stromstedt, M. et al. (1996) Arch. Biochem. Biophys. 329:73-81.

Takahashi, S., et al. (1998) Proc. Natl. Acad. Sci. USA 95:9879-9884.

U.S. Pat. No. 3,095,355

U.S. Pat. No. 5,550,316

U.S. Pat. No. 5,279,833

U.S. Pat. No. 5,872,011

U.S. Pat. No. 5,286,634

U.S. Pat. No. 5,834,228

U.S. Pat. No. 5,322,933

U.S. Pat. No. 2,868,691

U.S. Pat. No. 5,741,957

U.S. Pat. No. 4,196,265

U.S. Pat. No. 4,244,946

U.S. Pat. No. 4,554,101

U.S. Pat. No. 4,683,202

U.S. Pat. No. 4,736,866

U.S. Pat. No. 5,162,215

U.S. Pat. No. 5,234,933

U.S. Pat. No. 5,260,203

U.S. Pat. No. 5,651,964

U.S. Pat. No. 5,614,396

U.S. Pat. No. 5,625,125

U.S. Pat. No. 5,326,902

U.S. Pat. No. 5,399,346

U.S. Pat. No. 5,489,742

U.S. Pat. No. 5,734,033

U.S. Pat. No. 5,573,933

U.S. Pat. No. 5,646,008

U.S. Pat. No. 5,627,158

U.S. Pat. No.5,641,484

U.S. Pat. No. 5,643,567

U.S. Pat. No. 5,645,999

U.S. Pat. No. 5,648,061

Venkatramesh, M. & Nes, W. D. (1995) Arch. Biochem. Biophys. 324:189-99.

Venkatramesh et al., (1996) Biochim. Biophys. Acta 1299:313-24.

Vermilion, J. L. & Coon, M. J. (1978) J. Biol. Chem. 253:8812-9

Wang et al., Cell 86:435-444 (1996).

Wetmur & Davidson (1968) J. Mol. Biol. 31:349-370.

Wieser et al., EMBO J. 14:2199-2208 (1995).

Wrana et al., Cell 71:1003-1014 (1992).

Xu, S. H., et al. (1988) J. Chromatogr. 452:377-98.

Yoshida, Y. & Aoyama, Y. (1991) Biochem. Soc. Trans. 19:778-82.

Yoshida, Y., et al. (1997) J. Biochem. (Tokyo) 122:1122-8.

Yoshida et al., J. Biochem. 122:1122-1128 (1997)

Yoshida, Y. & Aoyama, Y. (1987) Biochem. Pharmacol. 36:229-35.

Zimmer et al., Peptides 1992, pp.393-394, ESCOM Science Publishers, B. V., 1993.

What is claimed is:

1. An isolated biologically active MT CYP51 polypeptide.

2. The polypeptide of claim 1, further characterized as comprising the amino acid sequence of any of SEQ ID NO's:2, 4, 6, 8 and 10.

3. The polypeptide of claim 1, modified to be in detectably labeled form.

4. The polypeptide of claim 1, further characterized as having crystalline form.

5. An isolated antibody capable of specifically binding to the polypeptide of claim 1.

6. The antibody of claim 5 which is a monoclonal antibody.

7. The antibody of claim 5 which is a polyclonal antibody.

8. A hybridoma cell line which produces the monoclonal antibody of claim 6.

9. An isolated antibody capable of neutralizing the biological activity of the polypeptide of claim 1.

10. The antibody of claim 9 which is a monoclonal antibody.

11. The antibody of claim 9 which is a polyclonal antibody.

12. A hybridoma cell line which produces the monoclonal antibody of claim 10.

13. An isolated nucleic acid molecule encoding a biologically active MT CYP51 polypeptide.

14. The nucleic acid molecule of claim 13, wherein the encoded MT CYP51 polypeptide comprises the amino acid sequence of any of SEQ ID NO's:2, 4, 6, 8 and 10.

15. The nucleic acid molecule of claim 14, further defined as comprising the MT CYP51-encoding nucleic acid sequence of any of SEQ ID NO's:1, 3, 5, 7 and 9.

16. The nucleic acid molecule of claim 13, further defined as a DNA segment.

17. The nucleic acid molecule of claim 15, further characterized as an isolated nucleic acid molecule selected from the group consisting of:
(a) an isolated nucleic acid molecule which hybridizes to the nucleic acid sequence given herein as any of SEQ ID NO's:1, 3, 5, 7 and 9 under wash stringency conditions represented by a wash solution having about 200 mM salt concentration and a wash temperature of at least about 45° C., and which encodes an MT CYP51 polypeptide; and
(b) an isolated nucleic acid molecule differing from the isolated nucleic acid molecule of (a) above in nucleotide sequence due to the degeneracy of the genetic code, and which encodes an MT CYP51 polypeptide encoded by the isolated nucleic acid of (a) above.

18. The nucleic acid molecule of claim 13, further comprising a promoter sequence.

19. The nucleic acid molecule of claim 13, further comprising a recombinant vector.

20. The nucleic acid molecule of claim 19, wherein the vector is a recombinant expression vector.

21. A recombinant host cell comprising the nucleic acid molecule of claim 13.

22. The recombinant host cell of claim 21, wherein the host cell is a procaryotic cell.

23. The recombinant host cell of claim 21, wherein the host cell is a eukaryotic cell.

24. A method of preparing an MT CYP51 polypeptide, comprising: transforming a cell with the nucleic acid molecule of claim 13 to produce MT CYP51 under conditions suitable for the expression of said polypeptide.

25. A process of producing an antibody immunoreactive with a MT CYP51 polypeptide, the process comprising steps of:
(a) transfecting a recombinant host cell with the nucleic acid molecule of claim 13, which encodes an MT CYP51 polypeptide;
(b) culturing the host cell under conditions sufficient for expression of the polypeptide;
(c) recovering the polypeptide; and
(d) preparing the antibody to the polypeptide.

26. The process of claim 25, wherein the polypeptide is any of SEQ ID NO's:2, 4, 6, 8 and 10.

27. The process of claim 25, wherein the nucleic acid molecule is any of SEQ ID NO's:1, 3, 5, 7 and 9.

28. An antibody produced by the process of claim 25.

29. A process of detecting an MT CYP51 polypeptide, the process comprising immunoreacting the polypeptide with an antibody prepared according to the process of claim 25 to form an antibody-polypeptide conjugate; and detecting the conjugate.

30. A process of detecting in a sample an RNA that encodes the MT CYP51 polypeptide encoded by the nucleic acid molecule of claim 13, said process comprising the steps of:
(a) contacting said sample under hybridizing conditions with the nucleic acid molecule of claim 13 to form a duplex; and
(b) detecting the duplex.

31. A process of detecting a DNA molecule that encodes an MT CYP51 polypeptide, the process comprising the steps of:
(a) hybridizing DNA molecules with the nucleic acid molecule of claim 13 to form a duplex; and
(b) detecting the duplex.

32. An assay kit for detecting the presence of an MT CYP51 polypeptide in a biological sample, the kit comprising a first container containing a first antibody capable of immunoreacting with an MT CYP51 polypeptide of claim 1, wherein the first antibody is present in an amount sufficient to perform at least one assay.

33. The assay kit of claim 32, further comprising a second container containing a second antibody that immunoreacts with the first antibody.

34. The assay kit of claim 33, wherein the first antibody and the second antibody comprise monoclonal antibodies.

35. The assay kit of claim 33, wherein the first antibody is affixed to a solid support.

36. The assay kit of claim 33, wherein the first and second antibodies each comprise an indicator.

37. The assay kit of claim 36, wherein the indicator is a radioactive label or an enzyme.

38. An assay kit for detecting the presence, in biological samples, of an MT CYP51 polypeptide, the kit comprising a first container that contains a nucleic acid segment identical or complimentary to a segment of at least ten contiguous nucleotide bases of the nucleic acid segment of claim 13.

39. An assay kit for detecting the presence, in a biological sample, of an antibody immunoreactive with an MT CYP51 polypeptide, the kit comprising a first container containing an MT CYP51 polypeptide of claim 1 that immunoreacts with the antibody, with the polypeptide present in an amount sufficient to perform at least one assay.

40. A method of screening candidate compounds for an ability to modulate CYP51 biological activity, the method comprising the steps of:
(a) establishing replicate test and control samples that comprise a biologically active CYP51 polypeptide;
(b) administering a candidate compound to the test sample but not the control sample;
(c) measuring the biological activity of the CYP51 polypeptide in the test and the control samples; and
(d) determining that the candidate compound modulates CYP51 biological activity if the biological activity of the CYP51 polypeptide measured for the test sample is greater or less than the biological activity of the CYP51 polypeptide level measured for the control sample.

41. The method of claim 40, wherein the CYP51 polypeptide comprises a biologically active MT CYP51 polypeptide.

42. The method of claim 41, wherein the MT CYP51 polypeptide is further characterized as having crystalline form.

43. The method of claim 41, wherein the replicate test and control samples further comprise a cell that expresses the biologically active MT CYP51 polypeptide.

44. A recombinant cell line suitable for use in the method of claim 43.

45. A method of identifying modulators of CYP450 enzymes by rational drug design, the method comprising the steps of:
(a) designing a potential modulator for a CYP450 enzyme that will form non-covalent bonds with amino acids in the CYP450 enzyme substrate binding site based upon the crystal structure of a MT CYP51 polypeptide;
(b) synthesizing the modulator; and
(c) determining whether the potential modulator inhibits the activity of a CYP450 enzyme.

46. A method of treating tuberculosis in a vertebrate subject, the method comprising the step of administering to the vertebrate subject a therapeutically effective amount of a substance capable of inhibiting MT CYP51 activity in MT in the vertebrate subject, whereby treatment of tuberculosis is accomplished.

47. The method of claim 46, wherein the inhibiting substance comprises an azole inhibitor.

48. The method of claim 47, wherein the azole inhibitor comprises ketoconazole.

49. The method of claim 46, wherein the substance capable of inhibiting MT CYP51 activity in the vertebrate subject comprises an anti-MT CYP51 antibody.

50. The method of claim 49, wherein the anti-MT CYP51 antibody comprises a monoclonal antibody.

51. A method of modulating cholesterol synthesis in a vertebrate subject in which modulation of cholesterol synthesis is desirable, the method comprising the step of administering to the vertebrate subject an effective amount of a substance which preferentially modulates CYP45014DM biological activity, whereby modulation of cholesterol synthesis is accomplished.

52. The method of claim 51, wherein the vertebrate subject is a mammal.

53. The method of claim 52, wherein the mammal is a human.

54. A method of modulating spermatogenesis in a vertebrate subject in which modulation of spermatogenesis is desirable, the method comprising the step of administering to the vertebrate subject an effective amount of a substance which preferentially modulates CYP45014DM biological activity, whereby modulation of spermatogenesis is accomplished.

55. The method of claim 54, wherein the vertebrate is a mammal.

56. The method of claim 55, wherein the mammal is a human.
